(19) United States
(12) Patent Application Publication
(10) Pub. No.: US 2023/0420134 A1
(43) Pub. Date: Dec. 28, 2023

(54) CANCER DIAGNOSIS AND CLASSIFICATION BY NON-HUMAN METAGENOMIC PATHWAY ANALYSIS

(71) Applicant: Micronoma, Inc., San Diego, CA (US)

(72) Inventors: Stephen WANDRO, San Diego, CA (US); Eddie ADAMS, San Diego, CA (US); Sandrine MILLER-MONTGOMERY, San Diego, CA (US)

(21) Appl. No.: 18/252,709

(22) PCT Filed: Nov. 16, 2021
(86) PCT No.: PCT/US2021/059559

§ 371 Date: May 11, 2023

Related U.S. Application Data
(60) Provisional application No. 63/114,447, filed on Nov. 16, 2020.

(51) Int. Cl. G16H 50/20; G16B 30/10; G16B 40/00; G16H 20/40

(52) U.S. Cl. CPC G16H 50/20; G16B 30/10; G16B 40/00; G16H 20/40

(57) ABSTRACT

Provided are methods for the diagnosis and classification of cancer by non-human metagenomic pathway analysis.

### CROSS-REFERENCE

This application claims benefit of U.S. Provisional Patent Application No. 63/114,447, filed Nov. 16, 2020, which is entirely incorporated herein by reference

### BACKGROUND

Recent studies on diverse cancer types indicate that tumors possess an endogenous microbiome that may be harnessed for improved prognosis, diagnosis, therapeutic selection, and to enhance our understanding of intra-tumor biology. Thus far, reports have provided evidence for a tumor-unique microbiome in cancers of the breast, prostate, colon, brain, bone, skin, and pancreas. Just how microbes come to colonize tumors is an area of active debate, but it has been demonstrated that independent of etiology, cancer-specific microbial associations can be exploited for diagnostic purposes via sequencing-based detection of microbial nucleic acids. Indeed, Poore et al., have shown that detection of microbial DNA (mbDNA) fragments in patient plasma samples could correctly discriminate among various cancers and non-cancer samples (PMID: 32214244 and PCT WO 2020/093040).

In Poore et al., metagenomic shotgun sequencing data derived from total plasma cell-free DNA—which, perforce, contains a mixture of human cfDNA and microbial cfDNA—was computationally segregated according to whether the sequencing reads mapped to a human reference genome. All unmapped—i.e., non-human—reads were then classified down to the genus level using a fast k-mer mapping approach (Kraken, PMID: 24580807). The output of the Kraken analysis is a list of taxonomic classifications for the sequencing reads in a sample and the read counts associated with each taxonomic assignment. In Poore al., this paired data (genera and read counts) derived from HIV-negative, healthy donors and cancer cohorts (lung, prostate and melanoma) were used as the inputs for machine learning classification algorithms to identify features unique to each cancer type. One disadvantage of using taxonomy-based classification is that the taxonomy assignment, while useful for cancer classification, does not directly inform one of what, if any, cancer-specific biochemical capacities may be provided by the tumor-associated microbiota. Having a method that can both classify and diagnose cancers while also providing information pertaining to the presence/abundance of biochemical capacities could help elucidate how intratumoral microbiota contribute to tumor-specific biology by either providing or consuming tumor required or produced metabolites, respectively.

Other prior art that is relevant to this field is as follows: U.S. Publication No. 2018/0223338 describes using the solid tissue microbiome or salvia microbiome in identifying and diagnosing head and neck cancer; and U.S. Publication No. 2018/0258495A1 describes using the solid tissue microbiome or fecal microbiome to detect colon cancer, some kinds of mutations associated with colon cancer, and a kit to collect and amplify the corresponding microbes. PCT WO 2019/191649 describes using cell-free microbial DNA and machine learning models to distinguish subjects having advanced adenoma and/or colorectal cancer from healthy subjects, wherein the machine learning algorithms rely upon DNA sequence reads mapping to reference genomes as input for analysis.

### SUMMARY

The disclosure provided herein describes systems and methods capable of accurately diagnosing or determining the presence or lack thereof cancer and other diseases, its subtypes, and its likelihood to respond to certain therapies solely using nucleic acids of non-human origin from a tissue or liquid biopsy sample. Specifically, the present invention provides methods that may identify the presence and abundance of microbial functional genes (and fragments thereof) and biochemical pathways present in a biopsy sample (e.g., a liquid or tissue biopsy). In some cases, the microbial functional genes and biochemical pathways may be utilized to train one or more models and/or predictive models, described elsewhere herein. Such trained models may output a determination of the presence or lack thereof a subject's cancer or the likelihood of therapeutic response and/or efficacy when a subject receives a treatment.

The methods of the present invention disclosed herein provide a method to generate a diagnostic model capable of diagnosing and classifying cancer whilst also providing information pertaining to the presence and or abundance of biochemical capacities to elucidate intratumoral microbiota contributions to tumor-specific biology. In some cases, tumor-specific biology may pertain to how intratumoral microbiota contribute to consuming tumor required or produced metabolites. For example, pathway-based analysis may help illuminate microbe-catalyzed conversions of therapeutic small molecules, enzymatic activities which may alter the in vivo efficacy of said molecules. To give a specific example using a therapeutic case where microbial activity has been directly implicated—bacterial mediated deamination of the cytidine moiety in the chemotherapeutic gemcitabine: it has been shown that bacteria expressing a long isoform of cytidine deaminase (cdd) can convert the active form of gemcitabine to the less therapeutically efficacious 2′2-difluorodeoxyuridine (PMID: 28912244). With this as biochemical test case, the present invention disclosed herein is aimed to address the unmet need of diagnosing cancer in a subject by way of his/her circulating microbial DNA, as detailed by Poore et al., while simultaneously detecting the presence/absence or abundance of the cancer-associated isoform of cdd. In view of this example, in some embodiments, the methods disclosed herein may not be limited only to diagnosing cancer in a subject but also predicting that the subject, if found to harbor the long isoform of cdd would likely not respond to gemcitabine treatment.

Aspects of the disclosure provided herein, in some embodiments, comprise a method of determining the presence or lack thereof cancer of a subject. In some embodiments, the method comprises: (a) providing one or more sequencing reads of a subject's biological sample; (b) filtering the sequencing reads with a genome database to produce a set of filtered non-human sequencing reads; (c) translating the non-human sequencing reads to non-human proteins; (d) mapping the non-human proteins to a protein database, thereby producing a set of protein database associations; and (e) determining the presence or lack thereof cancer of the subject as an output to the trained model when the trained model is provided an input of the set of protein database associations. In some embodiments, the set of protein database associations comprises a set of functional genes, biochemical pathways, or any combination thereof. In some embodiments, the method further comprises decontaminating the filtered non-human sequencing reads prior to (c) to remove contaminant non-human sequencing reads. In some embodiments, translating is completed in silico. In some embodiments, the biological sample is a tissue, liquid biopsy, or any combination thereof. In some embodiments, the subject is human or a non-human mammal. In some embodiments, the biological sample comprises a nucleic acid composition, wherein the nucleic acid composition comprises DNA, RNA, cell-free DNA, cell-free RNA, exosomal DNA, exosomal RNA, or any combination thereof. In some embodiments, the genome database is a human genome database. In some embodiments, the trained model is trained with a set of functional gene and biochemical pathway abundances that are present or absent with a characteristic abundance for a cancer of interest. In some embodiments, the non-human sequences originate from bacterial, archaeal, fungal, viral, or any combination thereof origins of life. In some embodiments, the trained model is configured to determine a category or tissue-specific location of the cancer of the subject. In some embodiments, the trained model is configured to determine one or more types of cancer of the subject. In some embodiments, the trained model is configured to determine one or more subtypes of the cancer of the subject. In some embodiments, the trained model is configured to determine a stage of cancer of the subject, cancer prognosis of the subject, or any combination thereof. In some embodiments, the trained model is configured to determine the presence or lack thereof cancer at a low-stage (stage I or stage II) tumor. In some embodiments, the trained model is configured to determine an immunotherapy response of the second set of one or more subjects when the second set of one or more subjects are provided the immunotherapy. In some embodiments, the method further comprises outputting with the trained model a therapy for the subject to treat the subject's cancer, wherein the subject will respond with positive therapeutic efficacy when administered the therapeutic. In some embodiments, the cancer of the subject comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the liquid biopsy comprises: plasma, serum, whole blood, urine, cerebral spinal fluid, saliva, sweat, tears, exhaled breath condensate, or any combination thereof. In some embodiments, filtering comprises computationally filtering of the sequencing reads by bowtie2, Kraken, or any combination thereof programs. In some embodiments, the protein database is the UniRef database. In some embodiments, translating is accomplished by BLASTP, USEARCH, LAST, MMSeqs2, DIAMOND, or any combination thereof software packages. In some embodiments, the mapping of the non-human proteins to the biochemical pathways is accomplished by mapping non-human proteins to KEGG, MetaCyc, PANTHER Pathway, PathBank or any combination thereof databases. In some embodiments, the biochemical pathways are generated with the software package MinPath.

Aspects of the disclosure, in some embodiments, describe A method of providing a determination of the presence or lack thereof cancer of a subject, the method comprising: (a) sequencing a nucleic acid compositions of a subject's biological sample thereby generating sequencing reads; (b) filtering the sequencing reads with a genome database to produce a set of filtered non-human sequencing reads; (c) translating the non-human sequencing reads to non-human proteins; (d) mapping the non-human proteins to a protein database, thereby producing a set of protein database associations; and (e) providing a determination of the presence or lack thereof cancer of the subject as an output of a trained model when the trained model is provided an input of the set protein database associations. In some embodiments, the set of protein database associations comprises a set of functional genes, biochemical pathways, or any combination thereof. In some embodiments, the method further comprises decontaminating the filtered non-human sequencing reads prior to (c) to remove contaminant non-human sequencing reads. In some embodiments, translating is completed in silico. In some embodiments, the biological sample is a tissue, liquid biopsy sample, or any combination thereof. In some embodiments, the subject is human or a non-human mammal. In some embodiments, the biological sample comprises a nucleic acid composition, wherein the nucleic acid composition comprises DNA, RNA, cell-free DNA, cell-free RNA, exosomal DNA, exosomal RNA, or any combination thereof. In some embodiments, the genome database is a human genome database. In some embodiments, the trained model is trained with a set of functional gene and biochemical pathway abundances that are present or absent with a characteristic abundance for a cancer of interest In some embodiments, the non-human sequences originate from bacterial, archaeal, fungal, viral, or any combination thereof origins of life. In some embodiments, the trained model is configured to determine a category or tissue-specific location of the cancer of the subject. In some embodiments, the trained model is configured to determine one or more types of the cancer of the subject. In some embodiments, the trained model is configured to determine one or more subtypes of the cancer of the subject. In some embodiments, the trained model is configured to determine a stage of a cancer of the subject, cancer prognosis of the subject, or any combination thereof. In some embodiments, the trained model is configured to determine the presence or lack thereof a cancer at a low-stage (stage I or stage II) tumor. In some embodiments, the trained model is configured to determine an immunotherapy response of the subject when the subject is provided an immunotherapy. In some embodiments, the method further comprises outputting with the trained model a therapy for the subject to treat the subject's cancer, wherein the subject will respond with positive therapeutic efficacy when administered the therapy. In some embodiments, the cancer of the subject comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the liquid biopsy comprises: plasma, serum, whole blood, urine, cerebral spinal fluid, saliva, sweat, tears, exhaled breath condensate, or any combination thereof. In some embodiments, filtering comprises computationally filtering of the sequencing reads by bowtie2, Kraken, or any combination thereof programs. In some embodiments, the protein database is the UniRef database. In some embodiments, translating is accomplished by BLASTP, USEARCH, LAST, MMSeqs2, DIAMOND, or any combination thereof software packages. In some embodiments, the mapping of the non-human proteins to the biochemical pathways is accomplished by mapping non-human proteins to KEGG, MetaCyc, PANTHER Pathway, PathBank or any combination thereof databases. In some embodiments, the biochemical pathways are generated with the software package MinPath.

Aspects of the disclosure provided herein, in some embodiments, describe a method of training a model configured to determine the presence or lack thereof cancer of a subject, the method comprising: (a) providing a dataset comprising nucleic acid sequencing reads of a first set of one or more subjects' nucleic acid compositions and a corresponding one or more cancers of the first set of one or more subjects; (b) filtering the nucleic acid sequencing reads with a build of a genome database to generate non-human sequencing reads; (c) translating the non-human sequencing reads to non-human proteins; (d) mapping the non-human proteins to a protein database, thereby producing a set of protein database associations; and (e) training a model with the set of protein database associations and the corresponding one or more cancer states of the first set of one or more subjects, thereby generating a trained model configured to determine the presence or lack thereof cancer of a second set of one or more subjects. In some embodiments, the set of protein database associations comprises a set of functional genes, biochemical pathways, or any combination thereof. In some embodiments, the method further comprises decontaminating the filtered non-human sequencing reads prior to (c) to remove contaminant non-human sequencing reads. In some embodiments, translating is completed in silico. In some embodiments, the biological sample is a tissue, liquid biopsy sample or any combination thereof. In some embodiments, the subject is human or a non-human mammal. In some embodiments, the biological sample comprises a nucleic acid composition, wherein the nucleic acid composition comprises DNA, RNA, cell-free DNA, cell-free RNA, exosomal DNA, exosomal RNA, or any combination thereof. In some embodiments, the genome database is a human genome database. In some embodiments, the trained model is trained with a set of functional gene and biochemical pathway abundances that are present or absent with a characteristic abundance for a cancer of interest. In some embodiments, the non-human sequences originate from bacterial, archaeal, fungal, viral, or any combination thereof origins of life. In some embodiments, the trained model is configured to determine a category or tissue-specific location of the second set of one or more subjects' cancer. In some embodiments, the trained model is configured to determine one or more types of the second set of one or more subjects' cancer. In some embodiments, the trained model is configured to determine one or more subtypes of the second set of one or more subjects' cancer. In some embodiments, the trained model is configured to determine a stage of the second set of one or more subjects' cancer, cancer prognosis, or any combination thereof. In some embodiments, the trained is configured to determine the presence or lack thereof the second set of one or more subjects' cancer at a low-stage (stage I or stage II) tumor. In some embodiments, the trained model is configured to determine an immunotherapy response of the subject when the subject is provided an immunotherapy. In some embodiments, the method further comprises outputting with the trained model a therapy to treat the second set of one or more subjects' cancer, wherein the second set of one or more subjects will respond with positive therapeutic efficacy when administered the therapy. In some embodiments, the first and second set of one or more subjects' cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the liquid biopsy comprises: plasma, serum, whole blood, urine, cerebral spinal fluid, saliva, sweat, tears, exhaled breath condensate, or any combination thereof. In some embodiments, filtering comprises computationally filtering of the sequencing reads by bowtie2, Kraken, or any combination thereof programs. In some embodiments, the protein database is the UniRef database. In some embodiments, translating is accomplished by BLASTP, USEARCH, LAST, MMSeqs2, DIAMOND, or any combination thereof software packages. In some embodiments, the mapping of the non-human proteins to the biochemical pathways is accomplished by mapping non-human proteins to KEGG, MetaCyc, PANTHER Pathway, PathBank or any combination thereof databases. In some embodiments, the biochemical pathways are generated with the software package MinPath. In some embodiments, the dataset further comprises a corresponding previous or current treatment administered to the first set of one or more subjects. In some embodiments, the dataset further comprises a treatment efficacy of the first set of one or more subjects' previous or current treatment administration.

Aspects of the disclosure provided herein, in some embodiments, describes a computer-implemented method for utilizing a trained predictive model to provide a therapeutic treatment prediction for one or more subjects, the method comprising: (a) receiving a first set of one or more subjects' nucleic acid sequencing reads of a biological sample and corresponding cancer classification; (b) filtering the nucleic acid sequencing reads with a build of a genome database to generate non-human sequencing reads; (c) translating the non-human sequencing reads to non-human proteins; (d) mapping the non-human proteins to a protein database, thereby producing a set of protein database associations; and (e) utilizing a trained predictive model to provide a treatment prediction for the first set of one or more subjects when the set of protein database associations are provided as an input to the trained predictive model. In some embodiments, the trained predictive model is trained on a second set of one or more subjects' nucleic acid sequencing reads of a biological sample, corresponding cancer classification, corresponding treatment administered, corresponding treatment response, or any combination thereof. In some embodiments, the second set of one or more subjects are different than the first set of one or more subjects. In some embodiments, the set of protein database associations comprises a set of functional genes, biochemical pathways, or any combination thereof. In some embodiments, the method further comprising decontaminating the filtered non-human sequencing reads prior to (c) to remove contaminant non-human sequencing reads. In some embodiments, translating is completed in silico. In some embodiments, the biological sample is a tissue, liquid biopsy sample or any combination thereof. In some embodiments, the first and/or second set of one or more subjects are human or a non-human mammal. In some embodiments, the biological sample nucleic acid composition comprises DNA, RNA, cell-free DNA, cell-free RNA, exosomal DNA, exosomal RNA, or any combination thereof. In some embodiments, the genome database is a human genome database. In some embodiments, the non-human sequences originate from bacterial, archaeal, fungal, viral, or any combination thereof origins of life. In some embodiments, the treatment prediction comprises an immunotherapy response of the first set of one or more subjects when the first set of one or more subjects are administered an immunotherapy. In some embodiments, the treatment prediction comprises a therapeutic efficacy that the first set of one or more subjects will respond with positive efficacy. In some embodiments, the cancer classification comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the liquid biopsy comprises: plasma, serum, whole blood, urine, cerebral spinal fluid, saliva, sweat, tears, exhaled breath condensate, or any combination thereof. In some embodiments, filtering comprises computationally filtering of the sequencing reads by bowtie2, Kraken, or any combination thereof programs. In some embodiments, the protein database is the UniRef database. In some embodiments, translating is accomplished by BLASTP, USEARCH, LAST, MMSeqs2, DIAMOND, or any combination thereof software packages. In some embodiments, the mapping of the non-human proteins to the biochemical pathways is accomplished by mapping non-human proteins to KEGG, MetaCyc, PANTHER Pathway, PathBank or any combination thereof databases. In some embodiments, the biochemical pathways are generated with the software package MinPath.

Aspects of the disclosure provided herein, in some embodiments, comprise a method of changing a subject's cancer treatment with a trained predictive model. In some embodiments, the method comprises: (a) providing one or more sequencing reads of a subject's biological sample with cancer, cancer type, and treatment administered to treat the cancer; (b) filtering the sequencing reads with a genome database to produce a set of filtered non-human sequencing reads; (c) translating the non-human sequencing reads to non-human proteins; (d) mapping the non-human proteins to a protein database, thereby producing a set of protein database associations; and (e) changing the subject's cancer treatment when the treatment administered differs from a treatment recommendation outputted by a trained predictive model when inputted with the set of protein database associations. In some embodiments, the trained predictive model is trained on a second set of one or more subjects' nucleic acid sequencing reads of a biological sample, corresponding cancer classification, corresponding treatment administered, corresponding treatment response, or any combination thereof. In some embodiments, the second set of one or more subjects are different than the first set of one or more subjects. In some embodiments, the set of protein database associations comprises a set of functional genes, biochemical pathways, or any combination thereof. In some embodiments, the method further comprises decontaminating the filtered non-human sequencing reads prior to (c) to remove contaminant non-human sequencing reads. In some embodiments, translating is completed in silico. In some embodiments, the biological sample is a tissue, liquid biopsy sample or any combination thereof. In some embodiments, the subject is human or a non-human mammal. In some embodiments, the biological sample nucleic acid composition comprises DNA, RNA, cell-free DNA, cell-free RNA, exosomal DNA, exosomal RNA, or any combination thereof. In some embodiments, the genome database is a human genome database. In some embodiments, the non-human sequences originate from bacterial, archaeal, fungal, viral, or any combination thereof origins of life. In some embodiments, the treatment recommendation comprises an immunotherapy response of the subject when the subject is administered an immunotherapy. In some embodiments, the treatment recommendation comprises a therapeutic that the subject will respond with positive efficacy. In some embodiments, the subject's cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the liquid biopsy comprises: plasma, serum, whole blood, urine, cerebral spinal fluid, saliva, sweat, tears, exhaled breath condensate, or any combination thereof. In some embodiments, filtering comprises computationally filtering of the sequencing reads by bowtie2, Kraken, or any combination thereof programs. In some embodiments, the protein database is the UniRef database. In some embodiments, the translating is accomplished by BLASTP, USEARCH, LAST, MMSeqs2, DIAMOND, or any combination thereof software packages. In some embodiments, the mapping of the non-human proteins to the biochemical pathways is accomplished by mapping non-human proteins to KEGG, MetaCyc, PANTHER Pathway, PathBank or any combination thereof databases. In some embodiments, the biochemical pathways are generated with the software package MinPath.

Aspects disclosed herein provide a method of creating a diagnostic model for diagnosing cancer in a subject based on taxonomy-independent non-human functional gene abundances in a biological sample comprising: (a) sequencing nucleic acid compositions in the biological sample to generating sequencing reads; (b) filtering the sequencing reads with a build of a genome database to isolate non-human sequencing reads; (c) translating in silico a composition of non-human sequencing reads to identify non-human proteins represented in the non-human sequencing reads; (c) mapping the non-human proteins to a non-human protein database of non-human functional genes and biochemical pathways; (d) mapping the non-human proteins to a non-human protein database of non-human functional genes and biochemical pathways; (e) generating functional gene and biochemical pathway abundance tables with the non-human functional genes and biochemical pathways; (f) analyzing the biochemical pathway abundance tables with a trained machine learning algorithm; and (g) using an output of the trained machine learning algorithm to provide a diagnosis of a presence or absence of the cancer of the subject. In some embodiments, the biological sample is a tissue, liquid biopsy sample or any combination thereof. In some embodiments, the subject is human or a non-human mammal. In some embodiments, the nucleic acid composition comprises a total population of DNA, RNA, cell-free DNA (cfDNA), cell-free RNA (cfRNA), exosomal DNA, exosomal RNA or any combination thereof. In some embodiments, the genome database is a human genome database. In some embodiments, the output of the trained machine learning algorithm comprises an analysis of the functional gene and biochemical pathway abundance tables. In some embodiments, the trained machine learning algorithm is trained with a set of functional gene and biochemical pathway abundances that are known to be present with a characteristic abundance or absent in a cancer of interest. In some embodiments, the diagnostic model utilizes biochemical pathway abundance information from one or more of the following domains of life: bacterial, archaeal, and/or fungal. In some embodiments, the diagnostic model diagnoses a category or tissue-specific location of cancer. In some embodiments, the diagnostic model is used to diagnose one or more types of cancer in a subject. In some embodiments, the diagnostic model is used to diagnose one more subtypes of cancer in a subject. In some embodiments, the diagnostic model is used to predict the stage of cancer in a subject and/or predict cancer prognosis in the subject. In some embodiments, the diagnostic model is used to diagnose a type of cancer at a low-stage (stage I or stage II) tumor. In some embodiments, the diagnostic model is used to predict immunotherapy response of a subject. In some embodiments, the diagnostic model is utilized to select an optimal therapy for a particular subject. In some embodiments, the diagnostic model is utilized to longitudinally model a course of one or more cancers' response to a therapy and to then adjust a treatment regimen. In some embodiments, the diagnostic model diagnoses one or more of the following: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma. In some embodiments, the diagnostic model identifies and removes certain non-human features as contaminants termed noise, while selectively retaining other non-human features termed signal. In some embodiments, the liquid biopsy sample includes but is not limited to one or more of the following: plasma, serum, whole blood, urine, cerebral spinal fluid, saliva, sweat, tears, or exhaled breath condensate. In some embodiments, the filtering comprises computationally filtering of sequencing reads by bowtie2, Kraken programs or any combination thereof. In some embodiments, the protein database is the UniRef database. In some embodiments, the non-human protein database is queried to identify proteins represented in the non-human sequencing reads is performed with the software package DIAMOND. In some embodiments, the database of biochemical pathways is the KEGG or MetaCyc Database. In some embodiments, generating biochemical pathway abundance tables is performed with the software package MiniPath.

Aspects disclosed herein provide a method of creating a diagnostic model for diagnosing cancer in a subject based on taxonomy-independent non-human functional gene abundances in a biological sample, the method comprising: (a) sequencing nucleic acid compositions in the biological sample to generate sequencing reads; (b) filtering the sequencing reads with a build of a genome database to isolate non-human sequencing reads; (c) mapping the non-human sequencing reads to a database of sequenced genomes; (d) generating a plurality of mapped genomic coordinates between the non-human sequencing reads and the database of sequenced genomes; (e) using the plurality of mapped genomic coordinates to query a database of known non-human proteins to calculate an abundance; (f) mapping the non-human proteins to a database of functional genes and biochemical pathways; (g) generating a plurality of functional gene and biochemical pathway abundance tables; (h) analyzing the functional gene and biochemical pathway abundance tables with a trained machine learning algorithm; and (i) using an output of the trained machine learning algorithm analysis of the plurality of functional gene and biochemical pathway abundance tables to diagnose a presence or absence of the cancer of the subject. In some embodiments, the diagnostic model utilizes a biochemical pathway abundance information from one or more of the following domains of life: bacterial, archaeal, and/or fungal. In some embodiments, the biological sample is a tissue, liquid biopsy sample or any combination thereof. In some embodiments, the subject is human or a non-human mammal. In some embodiments, the nucleic acid composition comprises a total population of DNA, RNA, cell-free DNA (cfDNA), cell-free RNA (cfRNA), exosomal DNA, exosomal RNA or any combination thereof. In some embodiments, the genome database is a human genome database. In some embodiments, the output of the trained machine learning algorithm comprises an analysis of the plurality of functional gene and biochemical pathway abundance tables. In some embodiments, the trained machine learning algorithm is trained with a set of functional gene and biochemical pathway abundances that are known to be present with a characteristic abundance or absent in the cancer of interest. In some embodiments, the diagnostic model diagnoses a category or tissue-specific location of cancer. In some embodiments, the diagnostic model is used to diagnose one or more types of cancer in a subject. In some embodiments, the diagnostic model is used to diagnose one or more subtypes of cancer in a subject. In some embodiments, the diagnostic model is used to predict the stage of cancer in a subject and/or predict cancer prognosis in the subject. In some embodiments, the diagnostic model is used to diagnose a type of cancer at low-stage (stage I or stage II) tumor. In some embodiments, the diagnostic model is used to predict immunotherapy response of a subject. In some embodiments, the diagnostic model is utilized to select an optimal therapy for a particular subject. In some embodiments, the diagnostic model is utilized to longitudinally model a course of one or more cancers' response to a therapy and to then adjust a treatment regime. In some embodiments, the diagnostic model diagnoses one or more of the following: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma. In some embodiments, the diagnostic model identifies and removes certain non-human features as contaminants termed noise, while selectively retaining other non-human features termed signal. In some embodiments, the liquid biopsy includes but is not limited to one or more of the following: plasma, serum, whole blood, urine, cerebral spinal fluid, saliva, sweat, tears, or exhaled breath condensate. In some embodiments, filtering comprises computationally filtering of sequencing reads by botwie2, Kaken programs or any combination thereof. In some embodiments, the database of sequenced genomes is the Web of Life database. In some embodiments, the protein database is the UniRef database. In some embodiments, the database of biochemical pathways is the KEGG or MetaCyc database.

In some embodiments, the invention provides a method for broadly creating patterns of microbial functional gene presence or abundance (‘signatures’) that are associated with the presence and/or type of cancer using liquid biopsy samples. These ‘signatures’ can then be deployed to diagnose the presence, kind, and/or subtype of cancer in a human.

In some embodiments, the invention provides a method for broadly creating patterns of microbial functional gene or abundance that are associated with the presence and/or type of cancer using primary tumor tissues. These ‘signatures’ can then be deployed to diagnose the presence, kind, and/or subtype of cancer in a human using liquid biopsy samples from said human.

In some embodiments, the invention provides a method of broadly diagnosing disease in a mammalian subject comprising: detecting microbial presence or abundance in a liquid biopsy sample from the subject; determining that the detected microbial functional gene or abundance is different than the microbial functional gene or abundance in a normal liquid biopsy sample, and correlating the detected microbial functional gene or abundance with a known microbial functional gene or abundance for a disease, thereby diagnosing the disease.

In some embodiments, the invention provides a method of diagnosing the type of disease in a mammalian subject comprising: detecting microbial presence or abundance in a liquid biopsy sample from the subject; determining that the detected microbial functional gene or abundance is similar or different to the microbial functional gene or abundance in a population of cancer and/or healthy patients with previously studied liquid biopsy samples, and correlating the detected microbial functional gene or abundance with the most similar liquid biopsy samples in this cohort, thereby diagnosing the disease and/or kind of disease.

In some embodiments, the invention provides a method of predicting which subjects will respond or will not respond to a particular treatment for disease, wherein the disease is cancer, wherein the subject is human, wherein the treatment is immunotherapy, wherein the immunotherapy is a PD-1 blockade (e.g. nivolumab, pembrolizumab).

In embodiments, the invention provides a method of diagnosing disease, further comprising treating the disease in the subject based on the identified non-mammalian features of the disease, wherein the disease is cancer, wherein the non-mammalian features are microbial, wherein the subject is human.

In some embodiments, the invention provides a method of diagnosing disease, further comprising longitudinal monitoring of its non-mammalian features to indicate response to treating the disease, wherein the disease is cancer, wherein the non-mammalian features are microbial, wherein the subject is human.

In some embodiments, the invention provides an assay to measure the microbial functional gene or abundance in the specified tissue samples, thereby permitting diagnosis of the disease.

In some embodiments, the invention utilizes a diagnostic model based on a machine learning architecture. In some embodiments, the invention utilizes a diagnostic model based on a regularized machine learning architecture.

In some embodiments, the invention utilizes a diagnostic model based on an ensemble of machine learning architectures. In some embodiments, the invention identifies and selectively removes certain non-mammalian features as contaminants termed noise, while selectively retaining other non-mammalian features as non-contaminants termed signal, wherein non-mammalian features are microbial.

In some embodiments, the invention provides a method of diagnosing disease wherein microbial functional gene or abundance information is combined with additional information about the host (subject) and/or the host's (subject's) cancer to create a diagnostic model that has greater predictive performance than only having microbial functional gene or abundance information alone.

In some embodiments, the diagnostic model utilizes information in combination with microbial functional gene or abundance information from one or more of the following sources: cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, and/or methylation patterns of circulating tumor cell derived RNA.

In some embodiments, microbial functional gene or abundance is detected by nucleic acid detection of one or more of the following methods: metagenomic shotgun sequencing, targeted microbial sequencing, host whole genome sequencing, host transcriptomic sequencing, cancer whole genome sequencing, and cancer transcriptomic sequencing.

In some embodiments, the microbial nucleic acids are detected simultaneously with nucleic acids from the host and subsequently distinguished.

In some embodiments, the host nucleic acids are selectively depleted, and the microbial nucleic acids are selectively retained prior to measurement (e.g. sequencing) of a combined nucleic acid pool.

In some embodiments, the invention provides that the tissue is blood, a constituent of blood (e.g. plasma), or a tissue biopsy, wherein the tissue biopsy may be malignant or non-malignant.

In some embodiments, the microbial functional gene or abundance of the cancer is determined by measuring microbial functional gene or abundance in other locations of the host.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1B show an example diagnostic model training scheme incorporating a metagenomic functional profiling module to enable metagenomic function-based discovery of health and disease-associated microbial signatures. FIG. 1A illustrates an exemplary training structure of a diagnostic model. FIG. 1B illustrates the use of the trained model of FIG. 1A to provide a diagnosis of disease and a classification of disease state where the trained model of FIG. 1A is provided new subject data of unknown disease status, as described in some embodiments herein.

FIG. 2A-2B show example workflows for two metagenomic function computational pipelines. FIG. 2A illustrates an exemplary metagenomic workflow using the HUMAnN 2.0 pipeline to generate gene and pathway abundance tables that can be input into the machine learning model of FIG. 1A. FIG. 2B illustrates an exemplary metagenomic workflow using the WolTka pipeline to generate gene and pathway abundance tables that can be input into the machine learning model of FIG. 1A, as described in some embodiments herein.

FIG. 3 shows the breakdown of a study population for healthy, cancerous, and lung disease used in generating a predictive model.

FIGS. 4A-4B show the pathway classification of non-human cell-free DNA sequences with HUMAnN 2.0 (Humann) and Web of Life Toolkit App (Woltka), as described in some embodiments herein.

FIGS. 5A-5B show a detailed mean pathway importance for pathways identified by Woltka analysis of cancer vs. health and cancer vs. lung disease sequenced cf-mbDNA samples, as described in some embodiments herein.

FIGS. 6A-6D show the receiver operating characteristic curves and area under the curve analysis indicating the accuracy of the various trained predictive models, as described in some embodiments herein.

FIG. 7 shows a study population breakdown of cancer and lung disease subjects whereby such subjects' cell-free DNA nucleic acid genetic pathway data were used to train predictive models, as described in some embodiments herein.

FIGS. 8A-8D show receiver operative characteristic curves and the calculated area under the curve for each predictive models trained on subjects' known cancer stage and corresponding cell-free mbDNA nucleic acid genetic pathway data, and subjects' with lung disease cell-free mbDNA nucleic acid genetic pathway data.

FIG. 9 shows a diagram of a computer system, configured to implement the methods of the disclosure, as described in some embodiments herein.

### DETAILED DESCRIPTION

The disclosure provided herein, describes a method to accurately diagnose and/or determine the presence or lack thereof one or more subjects' one or more cancers, subtypes, and/or the cancers likelihood of therapy response. In some cases, the one or more subjects' may be human or non-human mammals. The methods described herein may utilize nucleic acids of non-human origin from a tissue or liquid biopsy sample. This may be achieved by identifying specific patterns of microbial functional units (i.e., proteins including, but not limited to, enzymes, transcription factors, and receptors). In some embodiments, exemplary microbial enzymes that can be used for disease classification are provided in Table 1 and their presence or abundances (‘a signature’) within a sample to assign a certain probability that (1) the individual has cancer, (2) the individual has a cancer from a particular body site, (3) the individual has a particular type of cancer, (4) a cancer, which may or may not be diagnosed at the time, has a high or low likelihood or responding to a particular cancer therapy, (5) a cancer, which may or may not be diagnosed at the time, is found to harbor microbial features (e.g. microbial antigens) that can be targeted for developing a personalized therapeutic to treat the subject's cancer, or any combination thereof probabilities. Other uses for such methods are reasonably imaginable and readily implementable to those skilled in the art.

TABLE 1
Exemplary Functional Genes Detected and Used for Disease Classification

| Category | GeneID | Gene name | Pathway |
|---|---|---|---|
| Amino Acid | 1.1.1.23 | histidinol dehydrogenase | Histidine metabolism |
| Metabolism | | | |
| Amino Acid | 1.1.1.3 | homoserine dehydrogenase | Glycine, serine and threonine |
| Metabolism | | | metabolism; Cysteine and |
| | | | methionine metabolism; |
| | | | Lysine biosynthesis |
| Amino Acid | 1.1.1.85 | 3-isopropylmalate | Valine, leucine and isoleucine |
| Metabolism | | dehydrogenase | biosynthesis |
| Amino Acid | 1.1.1.86 | ketol-acid reductoisomerase | Valine, leucine and isoleucine |
| Metabolism | | | biosynthesis; Pantothenate |
| | | | and CoA biosynthesis |
| Amino Acid | 1.3.1.12 | prephenate dehydrogenase | Phenylalanine, tyrosine and |
| Metabolism | | | tryptophan biosynthesis; |
| | | | Novobiocin biosynthesis |
| Amino Acid | 1.3.1.26 | dihydrodipicolinate | Lysine biosynthesis |
| Metabolism | | reductase | |
| Amino Acid | 1.4.1.1 | L-alanine dehydrogenase | Alanine, aspartate and |
| Metabolism | | | glutamate metabolism; |
| | | | Taurine and hypotaurine |
| | | | metabolism |
| Amino Acid | 1.4.1.13 | glutamate synthase large | Alanine, aspartate and |
| Metabolism | | and small subunit (NADPH) | glutamate metabolism; |
| | | | Nitrogen metabolism |
| Amino Acid | 1.5.1.2 | pyrroline-5-carboxylate | Arginine and proline |
| Metabolism | | reductase | metabolism |
| Amino Acid | 1.5.99.8 | proline dehydrogenase | Arginine and proline |
| Metabolism | | | metabolism |
| Amino Acid | 2.1.2.1 | serine | Glycine, serine and threonine |
| Metabolism | | hydroxymethyltransferase | metabolism; Methane |
| | | | metabolism; Cyanoamino |
| | | | acid metabolism; Glyoxylate |
| | | | and dicarboxylate |
| | | | metabolism |
| Amino Acid | 2.1.3.3 | ornithine | Arginine and proline |
| Metabolism | | carbamoyltransferase 1 | metabolism |
| Amino Acid | 2.3.1.1 | N-acetylglutamate synthase | Arginine and proline |
| Metabolism | | | metabolism |
| Amino Acid | 2.3.1.16 | acetyl-CoA acyltransferase | Fatty acid metabolism; |
| Metabolism | | anaerobic | Valine, leucine and isoleucine |
| | | | degradation; Fatty acid |
| | | | elongation; alpha-Linolenic |
| | | | acid metabolism; Geraniol |
| | | | degradation; Biosynthesis of |
| | | | unsaturated fatty acids; |
| | | | Benzoate degradation; |
| | | | Ethylbenzene degradation |
| Amino Acid | 2.3.1.30 | serine O-acetyltransferase | Cysteine and methionine |
| Metabolism | | | metabolism; Sulfur |
| | | | metabolism |
| Amino Acid | 2.3.3.13 | 2-isopropylmalate synthase | Valine, leucine and isoleucine |
| Metabolism | | | biosynthesis; Pyruvate |
| | | | metabolism |
| Amino Acid | 2.4.2.17 | ATP | Histidine metabolism |
| Metabolism | | phosphoribosyltransferase | |
| Amino Acid | 2.5.1.16 | Spermidine Synthase | Arginine and proline |
| Metabolism | | | metabolism; Glutathione |
| | | | metabolism; Cysteine and |
| | | | methionine metabolism; |
| | | | beta-Alanine metabolism |
| Amino Acid | 2.5.1.47 | cysteine synthase A | Cysteine and methionine |
| Metabolism | | | metabolism; Sulfur |
| | | | metabolism |
| Amino Acid | 2.5.1.48 | cystathionine gamma- | Cysteine and methionine |
| Metabolism | | synthase | metabolism; Sulfur |
| | | | metabolism; |
| | | | Selenocompound |
| | | | metabolism |
| Amino Acid | 2.5.1.54 | 3-deoxy-7- | Phenylalanine, tyrosine and |
| Metabolism | | phosphoheptulonate | tryptophan biosynthesis |
| | | synthase | |
| Amino Acid | 2.6.1.42 | branched-chain-amino-acid | Glucosinolate biosynthesis; |
| Metabolism | | transaminase | Valine, leucine and isoleucine |
| | | | degradation; Valine, leucine |
| | | | and isoleucine biosynthesis; |
| | | | Pantothenate and CoA |
| | | | biosynthesis |
| Amino Acid | 2.6.1.66 | valine-pyruvate | Valine, leucine and isoleucine |
| Metabolism | | aminotransferase | biosynthesis |
| Amino Acid | 2.7.1.39 | homoserine kinase | Glycine, serine and threonine |
| Metabolism | | | metabolism |
| Amino Acid | 2.7.1.71 | shikimate kinase I II | Phenylalanine, tyrosine and |
| Metabolism | | | tryptophan biosynthesis |
| Amino Acid | 2.7.2.11 | gamma-glutamyl kinase | Arginine and proline |
| Metabolism | | | metabolism |
| Amino Acid | 2.7.2.4 | aspartate kinase | Glycine, serine and threonine |
| Metabolism | | | metabolism; Cysteine and |
| | | | methionine metabolism; |
| | | | Lysine biosynthesis |
| Amino Acid | 2.7.2.8 | acetylglutamate kinase | Arginine and proline |
| Metabolism | | | metabolism |
| Amino Acid | 2.8.3.5 | Butyryl CoA Acetate CoA | Synthesis and degradation of |
| Metabolism | | Transferase | ketone bodies; Valine, |
| | | | leucine and isoleucine |
| | | | degradation; Butanoate |
| | | | metabolism |
| Amino Acid | 3.5.1.2 | L-glutaminase | D-Glutamine and D- |
| Metabolism | | | glutamate metabolism; |
| | | | Alanine, aspartate and |
| | | | glutamate metabolism; |
| | | | Arginine and proline |
| | | | metabolism; Nitrogen |
| | | | metabolism |
| Amino Acid | 3.5.3.11 | Agamintase | Arginine and proline |
| Metabolism | | | metabolism |
| Amino Acid | 3.5.4.1 | cytosine deaminase | Arginine and proline |
| Metabolism | | | metabolism; Pyrimidine |
| | | | metabolism |
| Amino Acid | 3.5.4.19 | phosphoribosyl-AMP | Histidine metabolism |
| Metabolism | | cyclohydrolase | |
| Amino Acid | 3.6.1.31 | phosphoribosyl-ATP | Histidine metabolism |
| Metabolism | | pyrophosphatase | |
| Amino Acid | 4.1.1.15 | glutamate decarboxylase A | Taurine and hypotaurine |
| Metabolism | | and B PLP-dependent | metabolism; Alanine, |
| | | | aspartate and glutamate |
| | | | metabolism; beta-Alanine |
| | | | metabolism; Butanoate |
| | | | metabolism; GABAergic |
| | | | synapse; Type I diabetes |
| | | | mellitus |
| Amino Acid | 4.1.1.17 | Ornithine Decarboxylase | Arginine and proline |
| | | | metabolism; Glutathione |
| Metabolism | | | metabolism |
| Amino Acid | 4.1.1.18 | lysine decarboxylase 1 | Lysine degradation; Tropane, |
| Metabolism | | | piperidine and pyridine |
| | | | alkaloid biosynthesis |
| Amino Acid | 4.1.1.19 | biosynthetic arginine | Arginine and proline |
| Metabolism | | decarboxylase PLP-binding | metabolism |
| Amino Acid | 4.1.1.48 | Indole-3-glycerol-phosphate | Phenylalanine, tyrosine and |
| Metabolism | | synthase | tryptophan biosynthesis |
| Amino Acid | 4.1.2.14 | KDPG Aldolase | Pentose phosphate pathway; |
| Metabolism | | | Pentose and glucuronate |
| | | | interconversions; Arginine |
| | | | and proline metabolism |
| Amino Acid | 4.1.2.5 | L-threonine aldolase | Glycine, serine and threonine |
| Metabolism | | | metabolism |
| Amino Acid | 4.1.3.27 | anthranilate synthase | Phenylalanine, tyrosine and |
| Metabolism | | | tryptophan biosynthesis |
| Amino Acid | 4.2.1.10 | 3-dehydroquinate | Phenylalanine, tyrosine and |
| Metabolism | | dehydratase | tryptophan biosynthesis |
| Amino Acid | 4.2.1.52 | dihydrodipicolinate synthase | Lysine biosynthesis |
| Metabolism | | | |
| Amino Acid | 4.2.1.9 | dihydroxy-acid dehydratase | Valine, leucine and isoleucine |
| Metabolism | | | biosynthesis; Pantothenate |
| | | | and CoA biosynthesis |
| Amino Acid | 4.2.3.1 | L-threonine synthase | Glycine, serine and threonine |
| Metabolism | | | metabolism; Vitamin B6 |
| | | | metabolism |
| Amino Acid | 4.2.3.5 | chorismate synthase | Phenylalanine, tyrosine and |
| Metabolism | | | tryptophan biosynthesis |
| Amino Acid | 4.3.1.19 | threonine ammonia-lyase | Glycine, serine and threonine |
| Metabolism | | | metabolism; Valine, leucine |
| | | | and isoleucine biosynthesis |
| Amino Acid | 4.3.2.1 | argininosuccinate lyase | Alanine, aspartate and |
| Metabolism | | | glutamate metabolism; |
| | | | Arginine and proline |
| | | | metabolism |
| Amino Acid | 4.4.1.15 | D-cysteine desulfhydrase | Cysteine and methionine |
| Metabolism | | | metabolism |
| Amino Acid | 5.1.1.13 | aspartate racemase | Alanine, aspartate and |
| Metabolism | | | glutamate metabolism |
| Amino Acid | 5.1.1.7 | Diaminopimelate epimerase | Lysine biosynthesis |
| Metabolism | | | |
| Amino Acid | 5.4.99.5 | chorismate mutase | Phenylalanine, tyrosine and |
| Metabolism | | | tryptophan biosynthesis |
| Amino Acid | 6.3.1.1 | aspartate-ammonia ligase | Alanine, aspartate and |
| | | | glutamate metabolism; |
| | | | Cyanoamino acid |
| Metabolism | | | metabolism; Nitrogen |
| | | | metabolism |
| Amino Acid | 6.3.1.2 | L-glutamine synthase | Alanine, aspartate and |
| Metabolism | | | glutamate metabolism; |
| | | | Arginine and proline |
| | | | metabolism; Glyoxylate and |
| | | | dicarboxylate metabolism; |
| | | | Nitrogen metabolism |
| Amino Acid | 6.3.2.13 | UDP-N-acetylmuramoyl-L- | Lysine biosynthesis; |
| Metabolism | | alanyl-D-glutamatemeso- | Peptidoglycan biosynthesis |
| | | diaminopimelate ligase | |
| Amino Acid | 6.3.4.4 | adenylosuccinate synthetase | Purine metabolism; Alanine, |
| Metabolism | | | aspartate and glutamate |
| | | | metabolism |
| Amino Acid | 6.3.4.5 | arginosuccinate synthase | Alanine, aspartate and |
| Metabolism | | | glutamate |
| | | | metabolism; Arginine and |
| | | | proline metabolism |
| Amino Acid | 6.3.5.5 | carbamoyl phosphate | Pyrimidine metabolism; |
| Metabolism | | synthetase small subunit | Alanine, aspartate and |
| | | glutamine amidotransferase | glutamate metabolism |
| Carbohydrate | 1.1.1.27 | L-Lactate Dehydrogenase | Glycolysis/Gluconeogenesis; |
| Metabolism | | | Pyruvate metabolism; |
| | | | Propanoate metabolism |
| Carbohydrate | 1.1.2.3 | L-Lactate Dehydrogenase | Pyruvate metabolism |
| Metabolism | | (cytochrome) | |
| Carbohydrate | 2.1.2.1 | serine | Glycine, serine and threonine |
| Metabolism | | hydroxymethyltransferase | metabolism; Methane |
| | | | metabolism; Cyanoamino |
| | | | acid metabolism; Glyoxylate |
| | | | and dicarboxylate |
| | | | metabolism |
| Carbohydrate | 2.2.1.1 | Transketolase | Pentose phosphate pathway; |
| Metabolism | | | Carbon fixation in |
| | | | photosynthetic organisms; |
| | | | Biosynthesis of ansamycins |
| Carbohydrate | 2.2.1.2 | Transaldolase | Pentose phosphate pathway |
| Metabolism | | | |
| Carbohydrate | 2.3.1.54 | Pyruvate-Formate Lyase | Pyruvate metabolism; |
| Metabolism | | | Propanoate metabolism; |
| | | | Butanoate metabolism |
| Carbohydrate | 2.3.3.1 | Si-Citrate Synthase | Citrate cycle (TCA cycle) |
| Metabolism | | | |
| Carbohydrate | 2.3.3.13 | 2-isopropylmalate synthase | Valine, leucine and isoleucine |
| Metabolism | | | biosynthesis; Pyruvate |
| | | | metabolism |
| Carbohydrate | 2.4.1.21 | Glycogen Synthase | Starch and sucrose |
| Metabolism | | | metabolism |
| Carbohydrate | 2.7.1.12 | Gluconokinase | Pentose phosphate pathway |
| Metabolism | | | |
| Carbohydrate | 2.7.1.15 | Ribokinase | Pentose phosphate pathway |
| Metabolism | | | |
| Carbohydrate | 2.7.1.56 | 1-Phosphofructokinase | Fructose and mannose |
| Metabolism | | | metabolism |
| Carbohydrate | 2.7.1.6 | Galactokinase | Galactose metabolism |
| Metabolism | | | |
| Carbohydrate | 2.7.2.15 | Propionate Kinase | Propanoate metabolism |
| Metabolism | | | |
| Carbohydrate | 2.7.2.7 | Butyrate Kinase | Butanoate metabolism |
| Metabolism | | | |
| Carbohydrate | 2.8.3.5 | Butyryl CoA Acetate CoA | Synthesis and degradation of |
| Metabolism | | Transferase | ketone bodies; Valine, |
| | | | leucine and isoleucine |
| | | | degradation; Butanoate |
| | | | metabolism |
| Carbohydrate | 3.2.1.15 | Pectinase (Pectinesterase) | Pentose and glucuronate |
| | | | interconversions; Starch and |
| Metabolism | | | sucrose metabolism |
| Carbohydrate | 3.2.1.20 | alpha-Glucosidase | Starch and sucrose |
| Metabolism | | | metabolism; Galactose |
| | | | metabolism |
| Carbohydrate | 3.2.1.23 | beta-D-galactosidase | Glycosaminoglycan |
| Metabolism | | | degradation; Other glycan |
| | | | degradation; Galactose |
| | | | metabolism; Sphingolipid |
| | | | metabolism; |
| | | | Glycosphingolipid |
| | | | biosynthesis - ganglio series |
| Carbohydrate | 3.2.1.31 | beta-D-glucuronidase | Glycosaminoglycan |
| Metabolism | | | degradation; Pentose and |
| | | | glucuronate |
| | | | interconversions; Starch and |
| | | | sucrose metabolism; |
| | | | Porphyrin and chlorophyll |
| | | | metabolism; Flavone and |
| | | | flavonol biosynthesis; |
| | | | Lysosome |
| Carbohydrate | 3.2.1.52 | beta-N-acetyl-D- | Glycosaminoglycan |
| Metabolism | | hexosaminide N- | degradation; Other glycan |
| | | acetylhexosaminohydrolase | degradation; Amino sugar |
| | | | and nucleotide sugar |
| | | | metabolism; |
| | | | Glycosphingolipid |
| | | | biosynthesis - globo series; |
| | | | Glycosphingolipid |
| | | | biosynthesis - ganglio series; |
| | | | Various types of N-glycan |
| | | | biosynthesis |
| Carbohydrate | 3.2.1.67 | Extracellular Exopectate | Pentose and glucuronate |
| Metabolism | | Hydrolase | interconversions; Starch and |
| | | | sucrose metabolism |
| Carbohydrate | 3.2.1.91 | Cellulase (Exoglucanase) | Starch and sucrose |
| Metabolism | | | metabolism |
| Carbohydrate | 3.5.1.25 | N-Acetylglucosamine-6- | Amino sugar and nucleotide |
| Metabolism | | Phosphate Deacetylase | sugar metabolism; Galactose |
| | | | metabolism |
| Carbohydrate | 4.1.1.15 | glutamate decarboxylase A | Taurine and hypotaurine |
| Metabolism | | and B PLP-dependent | metabolism; Alanine, |
| | | | aspartate and glutamate |
| | | | metabolism; beta-Alanine |
| | | | metabolism; Butanoate |
| | | | metabolism; GABAergic |
| | | | synapse; Type I diabetes |
| | | | mellitus |
| Carbohydrate | 4.1.1.41 | Methylmalonyl-CaA | Propanoate metabolism |
| Metabolism | | decarboxylase | |
| Carbohydrate | 4.1.2.14 | KDPG Aldolase | Pentose phosphate pathway; |
| Metabolism | | | Pentose and glucuronate |
| | | | interconversions; Arginine |
| | | | and proline metabolism |
| Carbohydrate | 4.2.1.12 | Phosphogluconate | Pentose phosphate pathway |
| Metabolism | | dehydratase | |
| Carbohydrate | 4.2.2.2 | Extracellular Endopectate | Pentose and glucuronate |
| Metabolism | | Lyase | interconversions |
| Carbohydrate | 5.3.1.12 | Glucuronate Isomerase | Pentose and glucuronate |
| Metabolism | | | interconversion |
| Carbohydrate | 5.3.1.25 | Fucose Isomerase | Fructose and mannose |
| Metabolism | | | metabolism |
| Carbohydrate | 5.3.1.4 | Arabinose Isomerase | Pentose and glucuronate |
| Metabolism | | | interconversions |
| Carbohydrate | 5.3.1.5 | Xylose Isomerase | Pentose and glucuronate |
| Metabolism | | | interconversions; Fructose |
| | | | and mannose metabolism |
| Carbohydrate | 5.3.1.8 | Mannose-6-Phosphate | Fructose and mannose |
| Metabolism | | Isomerase | metabolism; Amino sugar |
| | | | and nucleotide sugar |
| | | | metabolism |
| Carbohydrate | 6.3.1.2 | L-glutamine synthase | Alanine, aspartate and |
| Metabolism | | | glutamate metabolism; |
| | | | Arginine and proline |
| | | | metabolism; Glyoxylate and |
| | | | dicarboxylate metabolism; |
| | | | Nitrogen metabolism |
| Energy Metabolism | 1.2.99.2 | Carbon Monoxide | Methane metabolism; |
| | | Dehydrogenase | Nitrotoluene degradation; |
| | | | Carbon fixation pathways in |
| | | | prokaryotes |
| Energy Metabolism | 1.4.1.13 | glutamate synthase large | Alanine, aspartate and |
| | | and small subunit (NADPH) | glutamate metabolism; |
| | | | Nitrogen metabolism |
| Energy Metabolism | 2.1.2.1 | serine | Glycine, serine and threonine |
| | | hydroxymethyltransferase | metabolism; Methane |
| | | | metabolism; Cyanoamino |
| | | | acid metabolism; Glyoxylate |
| | | | and dicarboxylate |
| | | | metabolism |
| Energy Metabolism | 2.2.1.1 | Transketolase | Pentose phosphate pathway; |
| | | | Carbon fixation in |
| | | | photosynthetic organisms; |
| | | | Biosynthesis of ansamycins |
| Energy Metabolism | 2.3.1.30 | serine O-acetyltransferase | Cysteine and methionine |
| | | | metabolism; Sulfur |
| | | | metabolism |
| Energy Metabolism | 2.5.1.47 | cysteine synthase A | Cysteine and methionine |
| | | | metabolism; Sulfur |
| | | | metabolism |
| Energy Metabolism | 2.5.1.48 | cystathionine gamma- | Cysteine and methionine |
| | | synthase | metabolism; Sulfur |
| | | | metabolism; |
| | | | Selenocompound |
| | | | metabolism |
| Energy Metabolism | 2.7.2.1 | Acetate Kinase | Taurine and hypotaurine |
| | | | metabolism; Methane |
| | | | metabolism; Carbon fixation |
| | | | pathways in prokaryotes |
| Energy Metabolism | 2.7.7.4 | sulfate adenylyltransferase | Selenocompound |
| | | subunit 2 | metabolism; Sulfur |
| | | | metabolism |
| Energy Metabolism | 3.5.1.1 | asparaginase | Cyanoamino acid |
| | | | metabolism; Nitrogen |
| | | | metabolism |
| Energy Metabolism | 3.5.1.2 | L-glutaminase | D-Glutamine and D- |
| | | | glutamate metabolism; |
| | | | Alanine, aspartate and |
| | | | glutamate metabolism; |
| | | | Arginine and proline |
| | | | metabolism; Nitrogen |
| | | | metabolism |
| Energy Metabolism | 6.3.1.1 | aspartate-ammonia ligase | Alanine, aspartate and |
| | | | glutamate metabolism; |
| | | | Cyanoamino acid |
| | | | metabolism; Nitrogen |
| | | | metabolism |
| Energy Metabolism | 6.3.1.2 | L-glutamine synthase | Alanine, aspartate and |
| | | | glutamate metabolism; |
| | | | Arginine and proline |
| | | | metabolism; Glyoxylate and |
| | | | dicarboxylate metabolism; |
| | | | Nitrogen metabolism |
| Energy Metabolism | 6.3.4.3 | Formyltetrahydrofolate | One carbon pool by folate; |
| | | synthetase | Carbon fixation pathways in |
| | | | prokaryotes |
| Glycan Biosynthesis | 2.3.1.129 | UDP-N-acetylglucosamine | Lipopolysaccharide |
| and Metabolism | | acyltransferase | biosynthesis |
| Glycan Biosynthesis | 2.7.8.13 | phospho-N-acetylmuramoyl- | Peptidoglycan biosynthesis |
| and Metabolism | | pentapeptide transferase | |
| Glycan Biosynthesis | 3.1.6.12 | N-acetyl-D-galactosamine-4- | Glycosaminoglycan |
| and Metabolism | | sulfate 4-sulfohydrolase | degradation |
| Glycan Biosynthesis | 3.1.6.14 | N-acetyl-D-glucosamine-6- | Glycosaminoglycan |
| and Metabolism | | sulfate 6-sulfohydrolase | degradation |
| Glycan Biosynthesis | 3.2.1.23 | beta-D-galactosidase | Glycosaminoglycan |
| and Metabolism | | | degradation; Other glycan |
| | | | degradation; Galactose |
| | | | metabolism; Sphingolipid |
| | | | metabolism; |
| | | | Glycosphingolipid |
| | | | biosynthesis - ganglio series |
| Glycan Biosynthesis | 3.2.1.24 | alpha-mannosidase | Other glycan degradation |
| and Metabolism | | | |
| Glycan Biosynthesis | 3.2.1.25 | Mannanase (beta- | Other glycan degradation; |
| and Metabolism | | mannosidase) | Lysosome |
| Glycan Biosynthesis | 3.2.1.31 | beta-D-glucuronidase | Glycosaminoglycan |
| and Metabolism | | | degradation; Pentose and |
| | | | glucuronate |
| | | | interconversions; Starch and |
| | | | sucrose metabolism; |
| | | | Porphyrin and chlorophyll |
| | | | metabolism; Flavone and |
| | | | flavonol biosynthesis; |
| | | | Lysosome |
| Glycan Biosynthesis | 3.2.1.52 | beta-N-acetyl-D- | Glycosaminoglycan |
| and Metabolism | | hexosaminide N- | degradation; Other glycan |
| | | acetylhexosaminohydrolase | degradation; Amino sugar |
| | | | and nucleotide sugar |
| | | | metabolism; |
| | | | Glycosphingolipid |
| | | | biosynthesis - globo series; |
| | | | Glycosphingolipid |
| | | | biosynthesis - ganglio series; |
| | | | Various types of N-glycan |
| | | | biosynthesis |
| Glycan Biosynthesis | 5.1.3.20 | ADP-L-glycero-D- | Lipopolysaccharide |
| and Metabolism | | mannoheptose-6-epimerase | biosynthesis |
| | | NAD(P)-binding | |
| Glycan Biosynthesis | 6.3.2.13 | UDP-N-acetylmuramoyl-L- | Lysine biosynthesis; |
| and Metabolism | | alanyl-D-glutamatemeso- | Peptidoglycan biosynthesis |
| | | diaminopimelate ligase | |
| Glycan Biosynthesis | 6.3.2.4 | D-alanineD-alanine ligase | D-Alanine metabolism; |
| and Metabolism | | | Peptidoglycan biosynthesis |
| Glycan Biosynthesis | 6.3.2.8 | UDP-N-acetylmuramateL- | D-Glutamine and D- |
| and Metabolism | | alanine ligase | glutamate metabolism; |
| | | | Peptidoglycan biosynthesis |
| Glycan Biosynthesis | 6.3.2.9 | UDP-N-acetylmuramoyl-L- | D-Glutamine and D- |
| and Metabolism | | alanineD-glutamate ligase | glutamate metabolism; |
| | | | Peptidoglycan biosynthesis |
| Lipid Metabolism | 2.3.1.16 | acetyl-CoA acyltransferase | Fatty acid metabolism; |
| | | anaerobic | Valine, leucine and isoleucine |
| | | | degradation; Fatty acid |
| | | | elongation; alpha-Linolenic |
| | | | acid metabolism; Geraniol |
| | | | degradation; Biosynthesis of |
| | | | unsaturated fatty acids; |
| | | | Benzoate degradation; |
| | | | Ethylbenzene degradation |
| Lipid Metabolism | 2.3.1.180 | beta-ketoacyl-acyl-carrier- | Fatty acid biosynthesis |
| | | protein synthase III | |
| Lipid Metabolism | 2.7.1.30 | Glycerol Kinase | Glycerolipid metabolism; |
| | | | PPAR signaling pathway; |
| | | | Plant-pathogen interaction |
| Lipid Metabolism | 2.8.3.5 | Butyryl CoA Acetate CoA | Synthesis and degradation of |
| | | Transferase | ketone bodies; Valine, |
| | | | leucine and isoleucine |
| | | | degradation; Butanoate |
| | | | metabolism |
| Lipid Metabolism | 3.2.1.23 | beta-D-galactosidase | Glycosaminoglycan |
| | | | degradation; Other glycan |
| | | | degradation; Galactose |
| | | | metabolism; Sphingolipid |
| | | | metabolism; |
| | | | Glycosphingolipid |
| | | | biosynthesis - ganglio series |
| Lipid Metabolism | 3.5.1.24 | Conjugated Bile Salt | Primary bile acid |
| | | Hydrolase | biosynthesis; Secondary bile |
| | | | acid biosynthesis |
| Metabolism of | 1.1.1.86 | ketol-acid reductoisomerase | Valine, leucine and isoleucine |
| Cofactors and | | | biosynthesis; Pantothenate |
| Vitamins | | | and CoA biosynthesis |
| Metabolism of | 2.1.1.64 | 3-demethylubiquinone-9 3- | Ubiquinone and other |
| Cofactors and | | methyltransferase | terpenoid-quinone |
| Vitamins | | | biosynthesis |
| Metabolism of | 2.5.1.9 | Riboflavin Synthase ?? | Riboflavin metabolism |
| Cofactors and | | Subunit | |
| Vitamins | | | |
| Metabolism of | 2.6.1.42 | branched-chain-amino-acid | Glucosinolate biosynthesis; |
| Cofactors and | | transaminase | Valine, leucine and isoleucine |
| Vitamins | | | degradation; Valine, leucine |
| | | | and isoleucine biosynthesis; |
| | | | Pantothenate and CoA |
| | | | biosynthesis |
| Metabolism of | 2.7.1.35 | Pyridoxal Kinase | Vitamin B6 metabolism |
| Cofactors and | | | |
| Vitamins | | | |
| Metabolism of | 2.7.1.89 | Thiamin Kinase | Thiamine metabolism |
| Cofactors and | | | |
| Vitamins | | | |
| Metabolism of | 2.7.8.26 | Cobalamin Synthase | Porphyrin and chlorophyll |
| Cofactors and | | | metabolism |
| Vitamins | | | |
| Metabolism of | 2.8.1.6 | Biotin Synthase | Biotin metabolism |
| Cofactors and | | | |
| Vitamins | | | |
| Metabolism of | 3.2.1.31 | beta-D-glucuronidase | Glycosaminoglycan |
| Cofactors and | | | degradation; Pentose and |
| Vitamins | | | glucuronate |
| | | | interconversions; Starch and |
| | | | sucrose metabolism; |
| | | | Porphyrin and chlorophyll |
| | | | metabolism; Flavone and |
| | | | flavonol biosynthesis; |
| | | | Lysosome |
| Metabolism of | 4.2.1.9 | dihydroxy-acid dehydratase | Valine, leucine and isoleucine |
| Cofactors and | | | biosynthesis; Pantothenate |
| Vitamins | | | and CoA biosynthesis |
| Metabolism of | 4.2.3.1 | L-threonine synthase | Glycine, serine and threonine |
| Cofactors and | | | metabolism; Vitamin B6 |
| Vitamins | | | metabolism |
| Metabolism of | 4.99.1.1 | Ferrochetalase | Porphyrin and chlorophyll |
| Cofactors and | | | metabolism |
| Vitamins | | | |
| Metabolism of | 4.99.1.3 | Cobalt Chelatase | Porphyrin and chlorophyll |
| Cofactors and | | | metabolism |
| Vitamins | | | |
| Metabolism of | 6.3.2.1 | Pantothenate Synthetase | beta-Alanine metabolism; |
| Cofactors and | | | Pantothenate and CoA |
| Vitamins | | | biosynthesis |
| Metabolism of | 6.3.2.17 | Folylpolyglutamate Synthase | Folate biosynthesis |
| Cofactors and | | | |
| Vitamins | | | |
| Metabolism of | 6.3.4.3 | Formyltetrahydrofolate | One carbon pool by folate; |
| Cofactors and | | synthetase | Carbon fixation pathways in |
| Vitamins | | | prokaryotes |
| Metabolism of | K03517 | Quinolinate Synthase | Nicotinate and nicotinamide |
| Cofactors and | | | metabolism |
| Vitamins | | | |
| Metabolism of Other | 1.4.1.1 | L-alanine dehydrogenase | Alanine, aspartate and |
| Amino Acids | | | glutamate metabolism; |
| | | | Taurine and hypotaurine |
| | | | metabolism |
| Metabolism of Other | 1.8.1.9 | thioredoxin reductase FAD- | Pyrimidine metabolism; |
| Amino Acids | | NADP-binding | Selenocompound |
| | | | metabolism |
| Metabolism of Other | 2.1.2.1 | serine | Glycine, serine and threonine |
| Amino Acids | | hydroxymethyltransferase | metabolism; Methane |
| | | | metabolism; Cyanoamino |
| | | | acid metabolism; Glyoxylate |
| | | | and dicarboxylate |
| | | | metabolism |
| Metabolism of Other | 2.5.1.16 | Spermidine Synthase | Arginine and proline |
| Amino Acids | | | metabolism; Glutathione |
| | | | metabolism; Cysteine and |
| | | | methionine metabolism; |
| | | | beta-Alanine metabolism |
| Metabolism of Other | 2.5.1.48 | cystathionine gamma- | Cysteine and methionine |
| Amino Acids | | synthase | metabolism; Sulfur |
| | | | metabolism; |
| | | | Selenocompound |
| | | | metabolism |
| Metabolism of Other | 2.7.2.1 | Acetate Kinase | Taurine and hypotaurine |
| Amino Acids | | | metabolism; Methane |
| | | | metabolism; Carbon fixation |
| | | | pathways in prokaryotes |
| Metabolism of Other | 2.7.7.4 | sulfate adenylyltransferase | Selenocompound |
| Amino Acids | | subunit 2 | metabolism; Sulfur |
| | | | metabolism |
| Metabolism of Other | 2.9.1.1 | L-Seryl-tRNASec selenium | Selenocompound |
| Amino Acids | | transferase | metabolism; Aminoacyl-tRNA |
| | | | biosynthesis |
| Metabolism of Other | 3.5.1.1 | asparaginase | Cyanoamino acid |
| Amino Acids | | | metabolism; Nitrogen |
| | | | metabolism |
| Metabolism of Other | 3.5.1.2 | L-glutaminase | D-Glutamine and D- |
| Amino Acids | | | glutamate metabolism; |
| | | | Alanine, aspartate and |
| | | | glutamate metabolism; |
| | | | Arginine and proline |
| | | | metabolism; Nitrogen |
| | | | metabolism |
| Metabolism of Other | 4.1.1.15 | glutamate decarboxylase A | Taurine and hypotaurine |
| Amino Acids | | and B PLP-dependent | metabolism; Alanine, |
| | | | aspartate and glutamate |
| | | | metabolism; beta-Alanine |
| | | | metabolism; Butanoate |
| | | | metabolism; GABAergic |
| | | | synapse; Type I diabetes |
| | | | mellitus |
| Metabolism of Other | 4.1.1.17 | Ornithine Decarboxylase | Arginine and proline |
| Amino Acids | | | metabolism; Glutathione |
| | | | metabolism |
| Metabolism of Other | 4.4.1.16 | selenocysteine lyase PLP- | Selenocompound |
| Amino Acids | | dependent | metabolism |
| Metabolism of Other | 5.1.1.1 | alanine racemase | D-Alanine metabolism |
| Amino Acids | | | |
| Metabolism of Other | 5.1.1.3 | glutamate racemase | D-Glutamine and D- |
| Amino Acids | | | glutamate metabolism |
| Metabolism of Other | 6.1.1.10 | methionyl-tRNA synthetase | Selenocompound |
| Amino Acids | | | metabolism; Aminoacyl-tRNA |
| | | | biosynthesis |
| Metabolism of Other | 6.3.1.1 | aspartate-ammonia ligase | Alanine, aspartate and |
| Amino Acids | | | glutamate metabolism; |
| | | | Cyanoamino acid |
| | | | metabolism; Nitrogen |
| | | | metabolism |
| Metabolism of Other | 6.3.1.8 | glutathionylspermidine | Glutathione metabolism |
| Amino Acids | | synthase | |
| Metabolism of Other | 6.3.2.1 | Pantothenate Synthetase | beta-Alanine metabolism; |
| Amino Acids | | | Pantothenate and CoA |
| | | | biosynthesis |
| Metabolism of Other | 6.3.2.3 | glutathione synthase | Glutathione metabolism |
| Amino Acids | | | |
| Metabolism of Other | 6.3.2.4 | D-alanineD-alanine ligase | D-Alanine metabolism; |
| Amino Acids | | | Peptidoglycan biosynthesis |
| Metabolism of Other | 6.3.2.8 | UDP-N-acetylmuramateL- | D-Glutamine and D- |
| Amino Acids | | alanine ligase | glutamate metabolism; |
| | | | Peptidoglycan biosynthesis |
| Metabolism of Other | 6.3.2.9 | UDP-N-acetylmuramoyl-L- | D-Glutamine and D- |
| Amino Acids | | alanineD-glutamate ligase | glutamate metabolism; |
| | | | Peptidoglycan biosynthesis |
| Metabolism of | 1.17.1.2 | 1-hydroxy-2-methyl-2-E- | Terpenoid backbone |
| Terpenoids and | | butenyl 4-diphosphate | biosynthesis |
| Polyketides | | reductase 4Fe—4S protein | |
| Metabolism of | 2.2.1.1 | Transketolase | Pentose phosphate pathway; |
| Terpenoids and | | | Carbon fixation in |
| Polyketides | | | photosynthetic organisms; |
| | | | Biosynthesis of ansamycins |
| Metabolism of | 2.3.1.16 | acetyl-CoA acyltransferase | Fatty acid metabolism; |
| Terpenoids and | | anaerobic | Valine, leucine and isoleucine |
| Polyketides | | | degradation; Fatty acid |
| | | | elongation; alpha-Linolenic |
| | | | acid metabolism; Geraniol |
| | | | degradation; Biosynthesis of |
| | | | unsaturated fatty acids; |
| | | | Benzoate degradation; |
| | | | Ethylbenzene degradation |
| Metabolism of | 2.5.1.10 | geranyltranstransferase | Terpenoid backbone |
| Terpenoids and | | | biosynthesis |
| Polyketides | | | |
| Metabolism of | 2.7.7.60 | 4-diphosphocytidyl-2C- | Terpenoid backbone |
| Terpenoids and | | methyl-D-erythritol synthase | biosynthesis |
| Polyketides | | | |
| Nucleotide | 1.1.1.205 | IMP dehydrogenase | Purine metabolism |
| Metabolism | | | |
| Nucleotide | 1.17.4.2 | anaerobic ribonucleoside- | Purine metabolism; |
| Metabolism | | triphosphate reductase | Pyrimidine metabolism |
| Nucleotide | 1.8.1.9 | thioredoxin reductase FAD- | Pyrimidine metabolism; |
| Metabolism | | NADP-binding | Selenocompound |
| | | | metabolism |
| Nucleotide | 2.4.2.1 | purine-nucleoside | Purine metabolism |
| Metabolism | | phosphorylase | |
| Nucleotide | 2.4.2.3 | uridine phosphorylase | Pyrimidine metabolism |
| Metabolism | | | |
| Nucleotide | 2.4.2.4 | thymidine phosphorylase | Pyrimidine metabolism |
| Metabolism | | | |
| Nucleotide | 2.7.4.14 | cytidylate kinase | Pyrimidine metabolism |
| Metabolism | | | |
| Nucleotide | 3.5.4.1 | cytosine deaminase | Arginine and proline |
| Metabolism | | | metabolism; Pyrimidine |
| | | | metabolism |
| Nucleotide | 3.6.1.13 | ADP-ribose pyrophosphatase | Purine metabolism |
| Metabolism | | | |
| Nucleotide | 4.1.1.23 | orotidine-5-phosphate | Pyrimidine metabolism |
| Metabolism | | decarboxylase | |
| Nucleotide | | phosphoribosylglycinamide | Purine metabolism |
| | | synthetase | |
| Metabolism | 6.3.4.13 | phosphoribosylamine- | |
| | | glycine ligase | |
| Nucleotide | 6.3.4.4 | adenylosuccinate synthetase | Purine metabolism; Alanine, |
| Metabolism | | | aspartate and glutamate |
| | | | metabolism |
| Nucleotide | 6.3.5.5 | carbamoyl phosphate | Pyrimidine metabolism; |
| Metabolism | | synthetase small subunit | Alanine, aspartate and |
| | | glutamine amidotransferase | glutamate metabolism |
| Translation | 2.9.1.1 | L-Seryl-tRNASec selenium | Selenocompound |
| | | transferase | metabolism; Aminoacyl-tRNA |
| | | | biosynthesis |
| Translation | 6.1.1.10 | methionyl-tRNA synthetase | Selenocompound |
| | | | metabolism; Aminoacyl-tRNA |
| | | | biosynthesis |
| Translation | 6.1.1.11 | Serine-tRNA ligase | Aminoacyl-tRNA biosynthesis |

### Sample Handling and Model Generation Methods

The methods described herein, may use nucleic acids of non-human origin to diagnose a condition (e.g., cancer) that has been traditionally thought to be a disease of the human genome. In some embodiments, methods may provide better clinical outcomes compared to a typical pathology report because since the methods described herein do not necessarily rely upon observed tissue structure, cellular atypia, or any other subjective measure traditionally used to diagnose cancer. In some cases, the methods may provide a high degree of sensitivity by focusing solely on microbial nucleic acid sources rather than modified human (i.e., cancerous) nucleic acid sources, which are modified often at extremely low frequencies in a background of ‘normal’ nucleic acid sources. In some embodiments, the methods disclosed herein may achieve such outcomes by either solid tissue and/or liquid biopsy samples, the latter of which may require minimal sample preparation and may be minimally invasive. In some embodiments, the liquid biopsy-based assay may overcome challenges posed by circulating tumor DNA (ctDNA) assays, which often suffer from sensitivity issues due to cell-free DNA (cfDNA) that originates from non-malignant human cells. In some instances, the liquid biopsy-based microbial assay may distinguish between cancer types, which ctDNA assays typically are not able to achieve, since most common cancer genomic aberrations are shared between cancer types (e.g., TP53 mutations, KRAS mutations). In some cases, the method described herein may constrain the size of the signatures, the method of which will be expected by someone knowledgeable in the art (e.g., regularized machine learning), the microbial assays may be made clinically available through the use of e.g. multiplexed quantitative polymerase chain reaction (qPCR), and targeted assay panels for multiplexed amplicon sequencing.

In some embodiments, the methods described herein may determine the presence or lack thereof cancer of a subject by utilizing trained models and/or trained predictive models, where the models and/or predictive models may comprise machine learning models trained on non-human functional gene and biochemical pathway abundances (i.e., non-human signatures) that can be deployed on real-time sequencing data or retrospective sequencing data (i.e., sequencing data from a database or repository). In some instances, the non-human signatures may comprise microbial signatures. In some cases, the methods for determining or diagnosing cancer of a subject may comprise a step of sequencing the nucleic acid compositions of a subject. Alternatively, the methods for determining or diagnosing cancer of a subject may comprise a step of accessing sequencing reads of a subject's biological sample nucleic acid compositions.

In some embodiments, the methods described herein may train a model by (a) taking a blood sample from a patient during a routine clinic visit; (b) preparing plasma or serum from that blood sample, extracting the nucleic acids within, and amplifying the sequences for specific microbial genes determined previously, by way of the previously trained machine learning model, to be useful signatures for diagnosing cancer; (c) obtaining a digital read-out of the presence and/or abundance of these microbial signatures; (d) normalizing the presence and/or abundance data on an adjacent computer or cloud computing infrastructure and feeding it into a previously trained machine learning model; and (e) reading out a prediction and a certain degree of confidence for how likely this sample (1) is associated with the presence or absence of cancer, (2) is associated with cancer of a particular type or bodily location, or (3) is associated with a high, intermediate, or low likelihood of response to a range of cancer therapies; and (f) using that sample's microbial information to continue training the machine learning model if additional information is later inputted by the user.

In some instances, the methods described herein may comprise a method of training a model configured to determine the presence or lack thereof cancer of the subject. In some cases, the method may comprise the steps of: (a) providing a dataset comprising nucleic acid sequencing reads of a first set of one or more subjects' nucleic acid compositions and a corresponding one or more cancers of the first set of one or more subjects; (b) filtering the nucleic acid sequencing reads with a build of a genome database to generate non-human sequencing reads; (c) translating the non-human sequencing reads to non-human proteins; (d) mapping the non-human proteins to a protein database, thereby producing a set of protein database associations; and (e) training a model with the set of protein database associations and the corresponding one or more cancer states of the first set of one or more subjects, thereby generating a trained model configured to determine the presence or lack thereof cancer of a second set of one or more subjects. In some instances, the set of protein database associations may comprise a set of functional genes, biochemical pathways, or any combination thereof, described elsewhere herein. In some instances, the method may further comprise decontaminating the filtered non-human sequencing reads prior to step (c) to remove contaminant non-human sequencing reads. In some cases, the contaminant non-human sequencing reads may be determined a prior or from a database of contaminant non-human sequencing reads determined from experimental data analysis. In some cases, the translating of step (c) may be completed in silico. In some instances, the method may in place of or in addition to step (a) comprise the step of sequencing nucleic acid compositions of the first set of one or more subjects. In some cases, the method may further comprise outputting with the trained model a therapy to treat the second set of one or more subjects' cancer, wherein the second set of one or more subjects will respond with positive therapeutic efficacy when administered the therapy. In some cases, the dataset may further comprise a corresponding previous or current treatment administered to the first set of one or more subjects. In some cases, the dataset may further comprise a treatment efficacy of the first set of one or more subjects' previous or current treatment administration.

In some cases, the first and/or second set of one or more subjects may be human or non-human mammal. In some cases, the biological sample may comprise a tissue, liquid biopsy sample or any combination thereof. In some cases, the biological sample may comprise a nucleic acid composition, where the nucleic acid composition may comprise DNA, RNA, cell-free RNA, exosomal DNA, exosomal RNA, or any combination thereof. In some cases the non-human sequences may originate from bacterial, archaeal, fungal, viral, or any combination thereof origins of life. In some instances, the liquid biopsy may comprise plasma, serum, whole blood, urine, cerebral spinal fluid, saliva, sweat, tears, exhaled breath condensate, or any combination thereof.

In some instances, the first and/or second set of one or more subjects may comprise cancer. In some cases, the cancer may comprise acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

In some cases, the trained model may be trained with a set of functional gene and biochemical pathway abundances that are present or absent with a characteristic abundance for a cancer of interest. In some instances, the trained model may be configured to determine one or more subtypes of the second set of one or more subjects' cancer. In some cases, the trained model may be configured to determine a stage of the second set of one or more subjects' cancer, cancer prognosis, or any combination thereof. In some instances, the trained model may be configured to determine the presence or lack thereof the second set of one or more subjects' cancer at a low-stage (stage I or stage II) tumor. In some cases, the trained model may be configured to determine an immunotherapy response of the subject when the subject is provided an immunotherapy. In some cases, the trained model may be configured to determine a category or tissue-specific location of the second set of one or more subjects' cancer. In some cases, the trained model may be configured to determine one or more types of the second set of one or more subjects' cancer.

In some instances the genome database may be a human genome database. In some cases step (b) of filtering may comprises computational filter of the sequencing reads by bowtie2, Kraken, or any combination thereof programs. In some instances, the protein database may be the UniRef database. In some cases, step (c) of translating may be accomplished by BLASTP, USEARCH, LAST, MMSeqs2, DIAMOND, or any combination thereof software packages. In some cases, step (d) of mapping of the non-human proteins to the biochemical pathways may be accomplished by mapping non-human proteins to KEGG, MetaCyc, PANTHER Pathway, PathBank, or any combination thereof databases. In some cases, the biochemical pathways may be generated with the software package MiniPath.

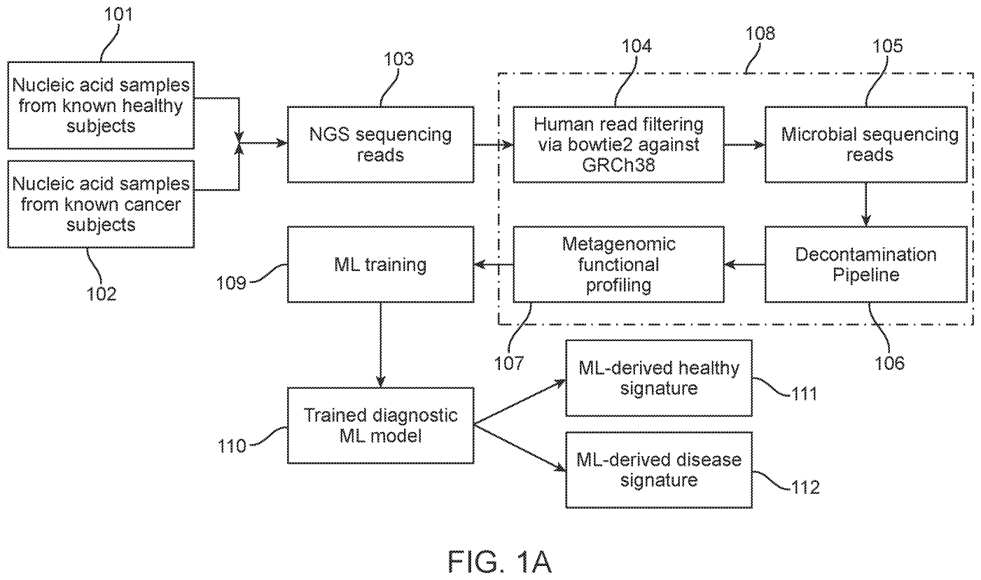

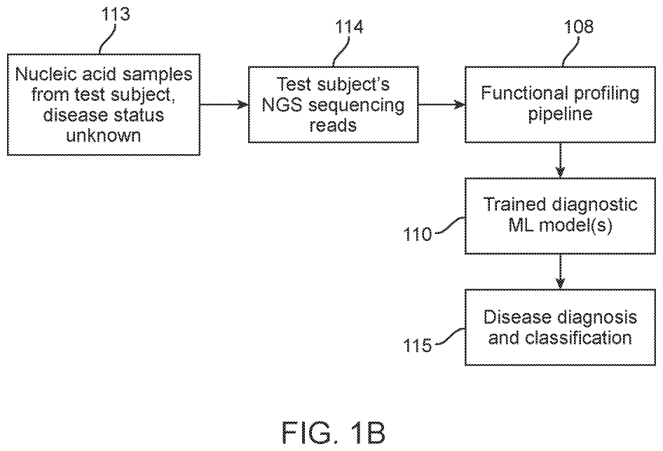

In some cases, the methods of the invention disclosed herein may comprise (a) sequencing the nucleic acid content of a liquid biopsy sample; and (b) generating a diagnostic model. In some embodiments, the sequencing method may comprise next-generation sequencing or long-read sequencing (e.g., nanopore sequencing) or a combination thereof. In some embodiments, the model 110 may comprise a diagnostic model. In some cases, the diagnostic model may comprise a trained machine learning algorithm 109 as shown in FIG. 1A. In some embodiments, the diagnostic model may be a regularized machine learning model. In some embodiments, the trained machine learning model algorithm may comprise a linear regression, logistic regression, decision tree, support vector machine (SVM), naïve bayes, k-nearest neighbors (kNN), k-Means, random forest algorithm model or any combination thereof. In some cases, the machine learning algorithm may comprise one or more machine learning algorithms.

In some embodiments, the machine learning algorithm 109 may be trained with nucleic acid sequencing data 103 derived from nucleic acids from a plurality of known healthy subjects 101 and a plurality of known cancer subjects 102. In some embodiments, the machine learning algorithm 109 may be trained with nucleic acid sequencing data 103 that has been processed through a metagenomic function bioinformatics pipeline 108 consisting of (a) computationally filtering all sequencing reads mapping to the human genome 104; (b) processing the remaining non-human microbial sequencing reads 105 through a decontamination pipeline 106 to remove sequences derived from common microbial contaminants; and (c) analyzing the remaining reads for their translated (i.e., protein) content 107. In some embodiments, computational filtering of all sequencing reads may be accomplished with bowtie2, Kraken programs or any equivalent thereof.

In some embodiments, the machine learning algorithm 109 may be trained resulting in a trained diagnostic model 110, where the trained diagnostic model may determine microbial signatures associated with and/or indicative of healthy subjects 111 and microbial signatures associated with/indicative of subjects with cancer 112.

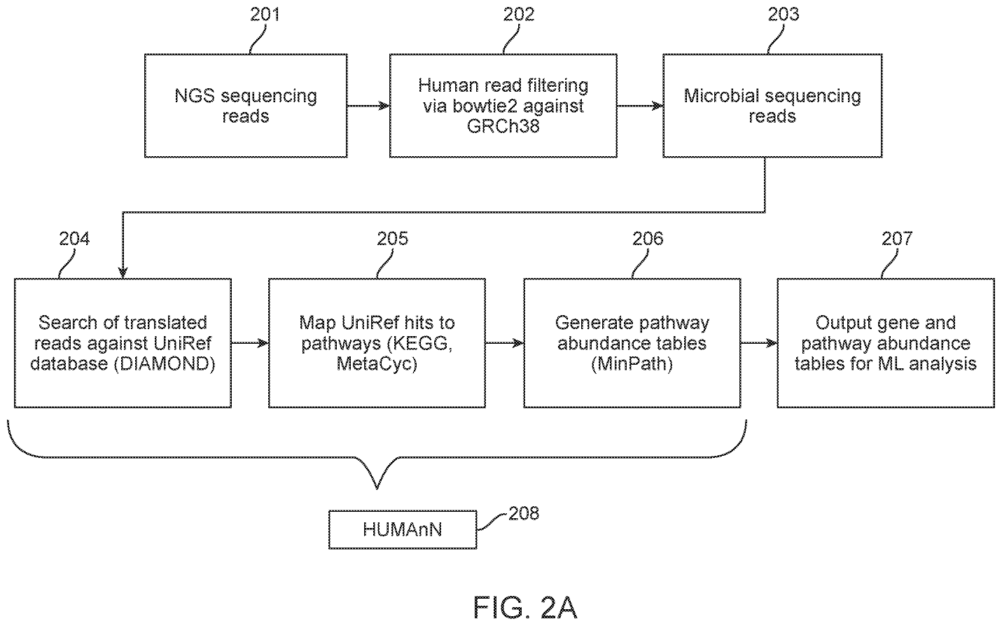

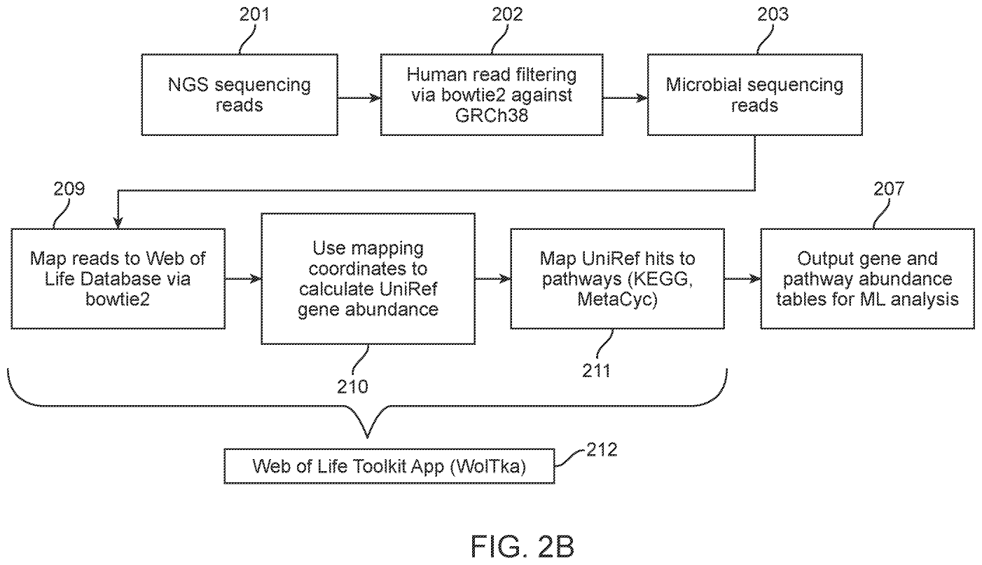

In some embodiments, the machine learning algorithm 109, as shown in FIG. 1A may additionally be trained with data pertaining to the abundance of functional microbial genes 207 (e.g., enzymes) in a sample or samples seen in FIG. 2A. In some embodiments, the abundance of functional microbial genes may be ascertained using the bioinformatics pipeline HUMAnN 208, as shown in FIG. 2A, including the steps of: (a) generating next generation sequencing reads from a subject's liquid biopsy (NGS) 201; (b) filtering human sequencing reads by bowtie, Kraken filtering methods or any equivalent thereof 202; (c) generating microbial sequencing as a result of filtering sequencing reads of (b) 203; (d) searching translated sequencing reads against a unitProt reference cluster (UniRef) database such as DIAMOND or an equivalent thereof 204; (e) mapping UniRef hits to pathways via Kyoto Encyclopedia of Genes and Genomes (Kegg), MetaCyc databases or any equivalent thereof 205; (f) generating pathway abundance tables with MiniPath; and (g) outputting pathway abundance tables for machine learning (ML) analysis 207.

In some embodiments, the abundance of functional microbial genes is ascertained using the bioinformatics pipeline Web of Life Toolkit App (WolTka) 212 or any equivalent thereof, as shown in FIG. 2B including the steps of: (a) generating next generation sequencing reads from a subject's liquid biopsy (NGS) 201; (b) filtering human sequencing reads by bowtie, kraken filtering methods or any equivalent thereof 202; (c) generating microbial sequencing as a result of filtering sequencing reads of (b) 203; (d) mapping sequencing reads of (c) to Web of Life Database with bowtie2 or any equivalent thereof read alignment tools 209 (e) using mapping coordinates from (d) to calculate UniREF gene abundance 210; (f) mapping UniRef hits to pathways with KEGG, MetaCyc or any equivalents thereof 211; and (g) outputting pathway abundance tables for machine learning (ML) analysis 207. The use of these bioinformatics pipelines and databases is not intended to be limiting but to serve as illustrations of the computational means by which one may arrive at microbial gene abundance data and therefore use of any substantial equivalent to the aforementioned bioinformatics.

Aspects disclosed herein provide a method of training a diagnostic model (FIG. 1A) comprising: (a) providing as a training data set (i) one or more subjects' one or more sequenced microbial functional gene abundances 108; (b) providing as a test set (i) one or more subjects' one or more sequenced microbial functional gene abundances 108; (c) training the diagnostic model on at least about a 10 to 90, 20 to 80, 30 to 70, 40 to 60, 50 to 50, 60 to 40, 70 to 30, 80 to 20, or 90 to 10 sample ratio of training to validation samples, respectively; and (d) evaluating the diagnostic accuracy of the diagnostic model. In some embodiments, the diagnosis made by the trained diagnostic model may

comprise a machine learning signature indicative of a healthy (i.e., cancer-free) subject 111, or a machine learning derived signature indicative of cancer-positive subject 112 as seen in FIG. 1A. In some embodiments, the trained diagnostic model may identify and remove the one more microbial or non-microbial nucleic acids classified as noise while selectively retaining other one or more microbial or non-microbial sequences termed signal.

### Diagnostic or Predictive Methods Utilizing Trained Models

In some embodiments, the trained diagnostic model 110 may be used to analyze the nucleic acid samples from subjects of unknown disease status 113 and provide a diagnosis of disease and, where applicable, classification of the state of that disease 115, as seen in FIG. 1B.

In some instances, the disclosure provided herein describes a method of determining the presence or lack thereof cancer of a subject. In some cases, the method may comprise the steps of: (a) providing one or more sequencing reads of a subject's biological sample; (b) filtering the sequencing reads with a genome database to produce a set of filtered non-human sequencing reads; (c) translating the non-human sequencing reads to non-human proteins; (d) mapping the non-human proteins to a protein database, thereby producing a set of protein database associations; and (e) determining the presence or lack thereof cancer of the subject as an output to the trained model when the trained model is provided an input of the set of protein database associations. In some instances, the set of protein database associations may comprise a set of functional genes, biochemical pathways, or any combination thereof, described elsewhere herein. In some instances, the method may further comprise decontaminating the filtered non-human sequencing reads prior to step (c) to remove contaminant non-human sequencing reads. In some cases, the contaminant non-human sequencing reads may be determined a prior or from a database of contaminant non-human sequencing reads determined from experimental data analysis. In some cases, the translating of step (c) may be completed in silico. In some instances, the method may in place of or in addition to step (a) comprise the step of sequencing nucleic acid compositions of the subjects. In some cases, the method may further comprise outputting with the trained model a therapy to treat the subject's cancer, where the subject will respond with positive therapeutic efficacy when administered the therapy.

In some cases, the subject may be human or non-human mammal. In some cases, the biological sample may comprise a tissue, liquid biopsy sample or any combination thereof. In some cases, the biological sample may comprise a nucleic acid composition, where the nucleic acid composition may comprise DNA, RNA, cell-free RNA, exosomal DNA, exosomal RNA, or any combination thereof. In some cases the non-human sequences may originate from bacterial, archaeal, fungal, viral, or any combination thereof origins of life. In some instances, the liquid biopsy may comprise plasma, serum, whole blood, urine, cerebral spinal fluid, saliva, sweat, tears, exhaled breath condensate, or any combination thereof.

In some instances, the subject may comprise cancer. In some cases, the cancer may comprise acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

In some cases, the trained model may be trained with a set of functional gene and biochemical pathway abundances that are present or absent with a characteristic abundance for a cancer of interest. In some instances, the trained model may be configured to determine one or more subtypes of the subject's cancer. In some cases, the trained model may be configured to determine a stage of the subject's cancer, cancer prognosis, or any combination thereof. In some instances, the trained model may be configured to determine the presence or lack thereof the subject's cancer at a low-stage (stage I or stage II) tumor. In some cases, the trained model may be configured to determine an immunotherapy response of the subject when the subject is provided an immunotherapy. In some cases, the trained model may be configured to determine a category or tissue-specific location of the subject's cancer. In some cases, the trained model may be configured to determine one or more types of the subject's cancer.

In some instances the genome database may be a human genome database. In some cases step (b) of filtering may comprises computational filter of the sequencing reads by bowtie2, Kraken, or any combination thereof programs. In some instances, the protein database may be the UniRef database. In some cases, step (c) of translating may be accomplished by BLASTP, USEARCH, LAST, MMSeqs2, DIAMOND, or any combination thereof software packages. In some cases, step (d) of mapping of the non-human proteins to the biochemical pathways may be accomplished by mapping non-human proteins to KEGG, MetaCyc, PANTHER Pathway, PathBank, or any combination thereof databases. In some cases, the biochemical pathways may be generated with the software package MiniPath.

In some instances, the disclosure provided herein describes a method of changing a subject's cancer treatment with a trained predictive model. In some cases, the method may comprise the steps of: (a) providing one or more sequencing reads of a subject's biological sample with cancer, cancer type, and treatment administered to treat the cancer; (b) filtering the sequencing reads with a genome database to produce a set of filtered non-human sequencing reads; (c) translating the non-human sequencing reads to non-human proteins; (d) mapping the non-human proteins to a protein database, thereby producing a set of protein database associations; and (e) changing the subject's cancer treatment when the treatment administered differs from the treatment recommendation outputted by a trained predictive model when inputted with the set of protein database associations. In some cases, the trained predictive model is trained on a second set of one or more subjects' nucleic acid sequencing reads of a biological sample, corresponding cancer classification, corresponding treatment administered, corresponding treatment response, or any combination thereof. In some cases, the second set of one or more subjects are different than the first set of one or more subjects. In some instances, the set of protein database associations may comprise a set of functional genes, biochemical pathways, or any combination thereof, described elsewhere herein. In some instances, the method may further comprise decontaminating the filtered non-human sequencing reads prior to step (c) to remove contaminant non-human sequencing reads. In some cases, the contaminant non-human sequencing reads may be determined a prior or from a database of contaminant non-human sequencing reads determined from experimental data analysis. In some cases, the translating of step (c) may be completed in silico. In some instances, the method may in place of or in addition to step (a) comprise the step of sequencing nucleic acid compositions of the subjects. In some cases, the method may further comprise outputting with the trained model a therapy to treat the subject's cancer, where the subject will respond with positive therapeutic efficacy when administered the therapy.

In some cases, the subject may be human or non-human mammal. In some cases, the biological sample may comprise a tissue, liquid biopsy sample or any combination thereof. In some cases, the biological sample may comprise a nucleic acid composition, where the nucleic acid composition may comprise DNA, RNA, cell-free RNA, exosomal DNA, exosomal RNA, or any combination thereof. In some cases the non-human sequences may originate from bacterial, archaeal, fungal, viral, or any combination thereof origins of life. In some instances, the liquid biopsy may comprise plasma, serum, whole blood, urine, cerebral spinal fluid, saliva, sweat, tears, exhaled breath condensate, or any combination thereof.

In some instances, the subject may comprise cancer. In some cases, the cancer may comprise acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

In some cases, the treatment recommendation comprises a therapeutic that the subject will respond with positive efficacy. In some cases, the treatment recommendation comprises an immunotherapy response of the subject when the subject is administered an immunotherapy.

In some instances the genome database may be a human genome database. In some cases step (b) of filtering may comprises computational filter of the sequencing reads by bowtie2, Kraken, or any combination thereof programs. In some instances, the protein database may be the UniRef database. In some cases, step (c) of translating may be accomplished by BLASTP, USEARCH, LAST, MMSeqs2, DIAMOND, or any combination thereof software packages. In some cases, step (d) of mapping of the non-human proteins to the biochemical pathways may be accomplished by mapping non-human proteins to KEGG, MetaCyc, PANTHER Pathway, PathBank, or any combination thereof databases. In some cases, the biochemical pathways may be generated with the software package MiniPath.

### Computer Systems

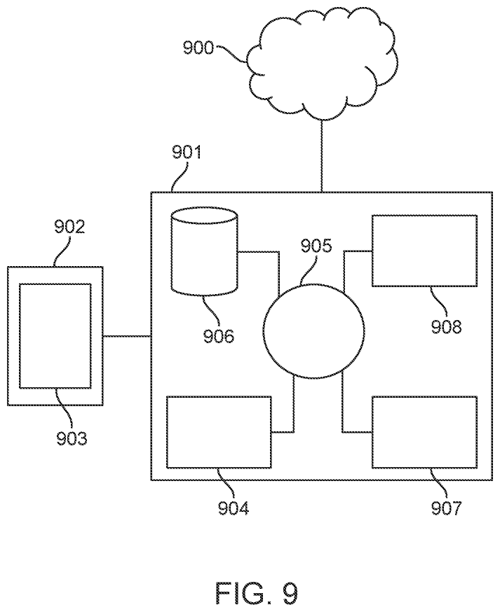

FIG. 9 shows a computer system 901 suitable for implementing and/or training the models and/or predictive models described herein. The computer system 901 may process various aspects of information of the present disclosure, such as, for example, subjects' sequences of a biological sample. The computer system 901 may be an electronic device. The electronic device may be a mobile electronic device.

The computer system 901 may comprise a central processing unit (CPU, also “processor” and “computer processor” herein) 905, which may be a single core or multi core processor, or a plurality of processor for parallel processing. The computer system 901 may further comprise memory or memory locations 904 (e.g., random-access memory, read-only memory, flash memory), electronic storage unit 906 (e.g., hard disk), communications interface 908 (e.g., network adapter) for communicating with one or more other devices, and peripheral devices 907, such as cache, other memory, data storage and/or electronic display adapters. The memory 904, storage unit 906, interface 908, and peripheral devices 907 are in communication with the CPU 905 through a communication bus (solid lines), such as a motherboard. The storage unit 906 may be a data storage unit (or a data repository) for storing data. The computer system 901 may be operatively coupled to a computer network (“network”) 400 with the aid of the communication interface 908. The network 400 may be the Internet, an internet and/or extranet, or an intranet and/or extranet that is in communication with the Internet. The network 400 may, in some case, be a telecommunication and/or data network. The network 400 may include one or more computer servers, which may enable distributed computing, such as cloud computing. The network 400, in some cases with the aid of the computer system 901, may implement a peer-to-peer network, which may enable devices coupled to the computer system 901 to behave as a client or a server.

The CPU 905 may execute a sequence of machine-readable instructions, which may be embodied in a program or software. The instructions may be directed to the CPU 905, which may subsequently program or otherwise configured the CPU 905 to implement methods of the present disclosure. Examples of operations performed by the CPU 905 may include fetch, decode, execute, and writeback.

The CPU 905 may be part of a circuit, such as an integrated circuit. One or more other components of the system 901 may be included in the circuit. In some cases, the circuit is an application specific integrated circuit (ASIC).

The storage unit 906 may store files, such as drivers, libraries and saved programs. The storage unit 906 may store one or more sequencing reads of one or more subjects' biological sample, cancer type if present, treatment administered to treat the cancer, treatment efficacy of the treatment administered, or any combination thereof. The computer system 901, in some cases may include one or more additional data storage units that are external to the computer system 901, such as located on a remote server that is in communication with the computer system 901 through an intranet or the internet.

Methods as described herein may be implemented by way of machine (e.g., computer processor) executable code stored on an electronic storage location of the computer device 901, such as, for example, on the memory 904 or electronic storage unit 906. The machine executable or machine-readable code may be provided in the form of software. During use, the code may be executed by the processor 905. In some instances, the code may be retrieved from the storage unit 906 and stored on the memory 904 for ready access by the processor 905. In some instances, the electronic storage unit 906 may be precluded, and machine-executable instructions are stored on memory 904.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 901, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage’ media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media include dynamic memory, such as main memory of such a computer platform. Tangible transmission media includes coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 902 that comprises a user interface (UI) 903 for viewing a therapeutic treatment outputted by a trained predictive model and/or recommendation or determination of a presence or lack thereof cancer for one or more subjects. Examples of UI's include, without limitation, a graphical user interface (GUI) and web-based user interface.

Methods and systems of the present disclosure can be implemented by way of one or more algorithms and with instructions provided with one or more processors as disclosed herein. An algorithm can be implemented by way of software upon execution by the central processing unit 905. The algorithm can be, for example, random forest, graphical models, support vector machine or other.

In some cases, the disclosure provided herein describes a computer-implemented method for utilizing a trained predictive model to provide a therapeutic treatment prediction for one or more subjects. In some instances, the method may comprise the steps of: (a) receiving a first set of one or more subjects' nucleic acid sequencing reads of a biological sample and corresponding cancer classification; (b) filtering the nucleic acid sequencing reads with a build of a genome database to generate non-human sequencing reads; (c) translating the non-human sequencing reads to non-human proteins; (d) mapping the non-human proteins to a protein database, thereby producing a set of protein database associations; and (e) utilizing a trained predictive model to provide a treatment prediction for the first set of one or more subjects when the set of protein database associates are provided as an input to the trained predictive model. In some cases, the method may further comprise the step of decontaminating the filtered non-human sequencing reads prior to (c) to remove contaminant non-human sequencing reads. In some instances, translating of step (c) may be completed in silico.

In some cases, the trained predictive model may be trained on a second set of one or more subjects' nucleic acid sequencing reads of a biological sample, corresponding cancer classification, corresponding treatment administered, corresponding treatment response, or any combination thereof. In some instances, the second set of one or more subjects may be different than the first set of one or more subjects. In some cases, set of protein database associations may comprises a set of functional genes, biochemical pathways, or any combination thereof. In some cases, the biological sample may comprise a tissue, liquid biopsy sample or any combination thereof. In some instances the liquid biopsy may comprises: plasma, serum, whole blood, urine, cerebral spinal fluid, saliva, sweat, tears, exhaled breath condensate, or any combination thereof. In some cases, the first set of one or more subjects may be human or a non-human mammal. In some instances, the biological sample nucleic acid composition may comprise DNA, RNA, cell-free DNA, cell-free RNA, exosomal DNA, exosomal RNA, or any combination thereof. In some instances, the genome database may be a human genome database. In some cases, the non-human sequences may originate from bacterial, archaeal, fungal, viral, or any combination thereof origins of life. In some instances, the treatment prediction may comprise an immunotherapy response of the first set of one or more subjects when the first set of one or more subjects are administered an immunotherapy. In some instances, the treatment prediction may comprise a therapeutic efficacy that the first set of one or more subjects will respond with positive efficacy. In some cases, the cancer classification may comprise comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

In some cases, filtering of step (b) may comprise computationally filtering of the sequencing reads by bowtie2, Kraken, or any combination thereof programs. In some cases, the protein database may be the UniRef database. In some instances, translating of step (c) may be accomplished by BLASTP, USEARCH, LAST, MMSeqs2, DIAMOND, or any combination thereof software packages. In some cases, mapping of the non-human proteins to the biochemical pathways of step (d) may be accomplished by mapping non-human proteins to KEGG, MetaCyc, PANTHER Pathway, PathBank or any combination thereof databases. In some cases, the biochemical pathways may be generated with the software package MinPath.

Although the above steps show a method of a system in accordance with an example, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the platform.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

### Example 1: Generating and Utilizing a Diagnostic Model Trained on Genetic Pathways for Disease Diagnosis and Classification

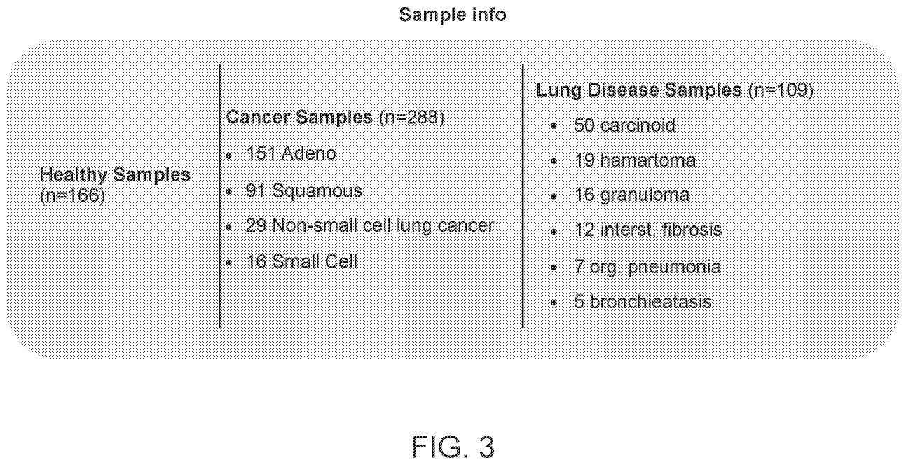

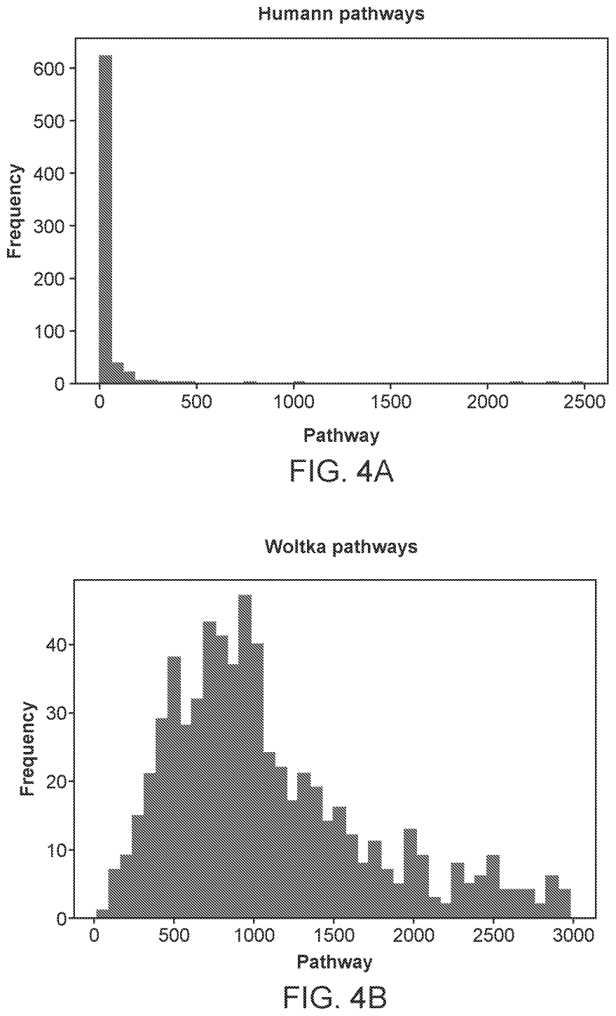

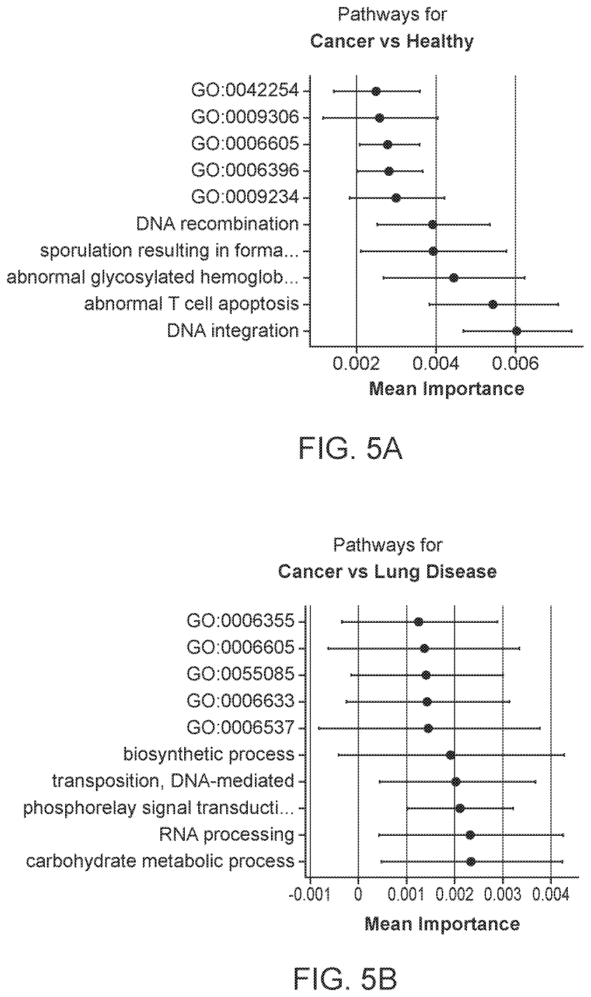

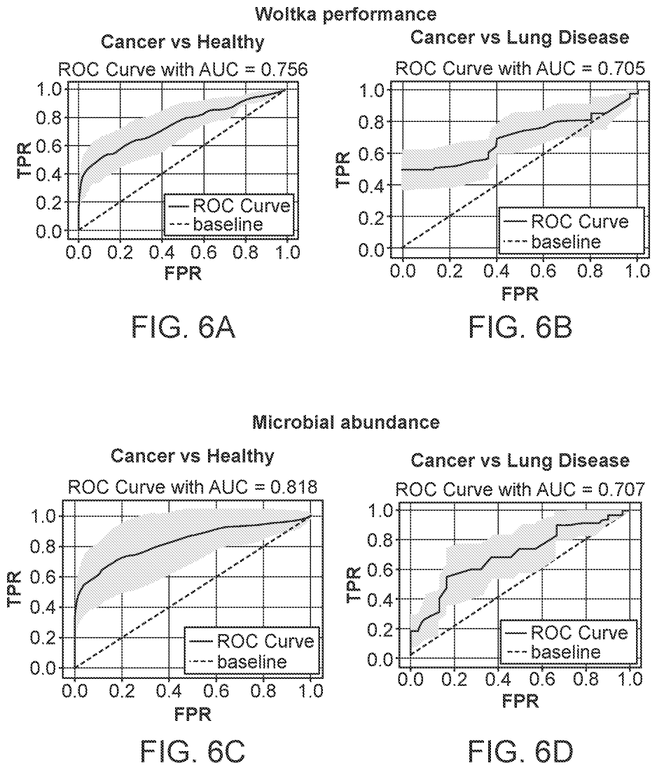

Diagnostic models configured to classify subjects categorically based on their non-mammalian pathway abundance as healthy, having lung cancer, or having lung disease were generated and tested. Cell-free DNA (cfDNA) sequencing libraries of 166 healthy, 288 lung cancer, and 109 lung disease subjects was obtained and further processed. Further breakdown of the sub cancer categories is referenced in FIG. 3. The cfDNA sequencing samples were then aligned with biochemical pathway classifications using both Web of Life Toolkit App (Woltka) and HUMAnN 3.0 (Humann) pipelines shown in FIGS. 4A-4B. Based upon this initial analysis, it was determined that the Woltka classified the samples into a more representative distribution of pathways than the Humann toolkit. From the Woltka classified pathways, the following gene ontology (GO) pathways were found to be the most important features for machine learning-based classifiers: GO:0055085: transmembrane transport; GO:0005975: carbohydrate metabolic process; GO:0006412: translation; GO:0006313: transposition, DNA-mediated; GO:0006355: regulation of transcription, DNA-templated; GO:0006260: DNA replication; GO:0006351: transcription, DNA-templated; and GO:0000160:phosphorelay signal transduction system. Other pathways identified to have importance in differentiating cancer vs. healthy and cancer vs. lung disease subjects can be seen in FIGS. 5A-5B. Microbial pathways identified via the WolTka pipeline in FIG. 2B were used as inputs to train the predictive models (e.g., a 10-fold cross validation Random forest), enabling differentiation of cancer vs. healthy and cancer vs. lung disease. The performance of each model, as represented by area under the receiver operating characteristics (AUC) analysis (FIGS. 6A-6B) can be compared to predictive models for cancer vs. healthy and cancer vs. lung disease trained on microbial taxonomy abundance shown in FIGS. 6C-D. It was found that the predictive model trained on the pathway importance as classified by the Woltka was able to differentiate cancer vs. healthy subjects with an AUC of 0.756 and cancer vs. lung disease with an AUC of 0.705 comparable to the AUC of 0.818 for cancer vs. healthy and 0.707 for cancer vs. lung disease of the microbial taxonomy trained predictive models.

### Example 2: Generating and Utilizing a Diagnostic Model Trained on Genetic Pathways for Determining Cancer Stage

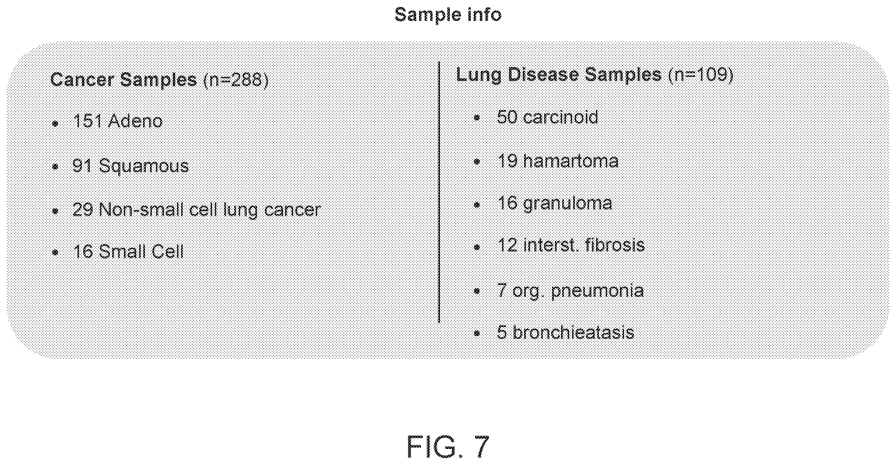

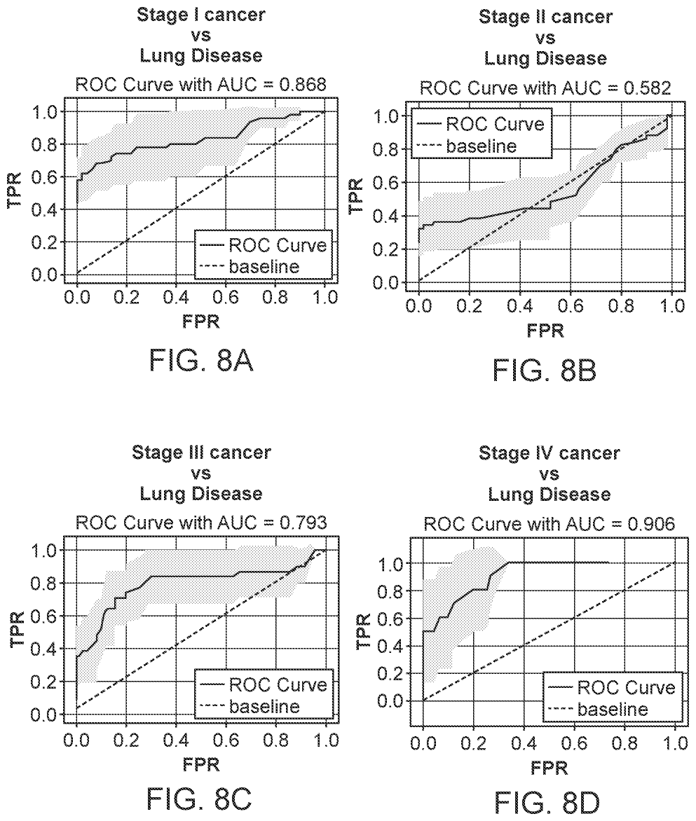

Diagnostic models configured to classify subjects' cancer stage based on non-mammalian pathway abundance in a background of a pathway abundance of lung disease were generated and tested. Cell-free DNA (cfDNA) sequencing data of subjects with cancer at varying stages in addition to subjects with lung disease were obtained. The sequencing data was comprised of 288 subjects with cancer at varying known stages and 109 subjects with lung disease, as shown in FIG. 7. A further breakdown of the cancer type and number of sub categories is shown in FIG. 7 as well. A plurality of Woltka classified pathways for the cf-mbDNA sequences were determined, as shown in Example 1, and used to train a Random Forest with 10-fold cross validation. Each trained Random Forest predictive model accuracy was then analyzed by area under the receiver operating characteristic curve (AUC) as shown in FIGS. 8A-8D. It was found that predictive models trained on the pathway importance as classified by the Woltka was able to differentiate stage 1 cancer vs. lung disease with an AUC of 0.868, stage 2 cancer vs. lung disease with an AUC of 0.582, stage 3 cancer vs. lung disease with an AUC of 0.793, and stage 4 cancer vs. lung disease with an AUC of 0.906.

### EMBODIMENTS

1. A method of determining the presence or lack thereof cancer of a subject, the method comprising:
   (a) providing one or more sequencing reads of a subject's biological sample;
   (b) filtering the sequencing reads with a genome database to produce a set of filtered non-human sequencing reads;
   (c) translating the non-human sequencing reads to non-human proteins;
   (d) mapping the non-human proteins to a protein database, thereby producing a set of protein database associations; and
   (e) determining the presence or lack thereof cancer of the subject as an output to the trained model when the trained model is provided an input of the set of protein database associations.
2. The method of embodiment 1, wherein the set of protein database associations comprises a set of functional genes, biochemical pathways, or any combination thereof.
3. The method of embodiment 1, further comprising decontaminating the filtered non-human sequencing reads prior to (c) to remove contaminant non-human sequencing reads.
4. The method of embodiment 1, wherein translating is completed in silico.
5. The method of embodiment 1, wherein the biological sample is a tissue, liquid biopsy, or any combination thereof.
6. The method of embodiment 1, wherein the subject is human or a non-human mammal.
7. The method of embodiment 1, wherein the biological sample comprises a nucleic acid composition, wherein the nucleic acid composition comprises DNA, RNA, cell-free DNA, cell-free RNA, exosomal DNA, exosomal RNA, or any combination thereof.
8. The method of embodiment 1, wherein the genome database is a human genome database.
9. The method of embodiment 1, wherein the trained model is trained with a set of functional gene and biochemical pathway abundances that are present or absent with a characteristic abundance for a cancer of interest.
10. The method of embodiment 1, wherein the non-human sequences originate from bacterial, archaeal, fungal, viral, or any combination thereof origins of life.
11. The method of embodiment 1, wherein the trained model is configured to determine a category or tissue-specific location of the cancer of the subject.
12. The method of embodiment 1, wherein the trained model is configured to determine one or more types of cancer of the subject.
13. The method of embodiment 12, wherein the trained model is configured to determine one or more subtypes of the cancer of the subject.
14. The method of embodiment 1, wherein the trained model is configured to determine a stage of cancer of the subject, cancer prognosis of the subject, or any combination thereof.
15. The method of embodiment 1, wherein the trained model is configured to determine the presence or lack thereof cancer at a low-stage (stage I or stage II) tumor.
16. The method of embodiment 1, wherein the trained model is configured to determine an immunotherapy response of the subject when the subject is provided the immunotherapy.
17. The method of embodiment 1, further comprising outputting with the trained model a therapy for the subject to treat the subject's cancer, wherein the subject will respond with positive therapeutic efficacy when administered the therapeutic.
18. The method of embodiment 1, wherein the cancer of the subject comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.
19. The method of embodiment 5, wherein the liquid biopsy comprises: plasma, serum, whole blood, urine, cerebral spinal fluid, saliva, sweat, tears, exhaled breath condensate, or any combination thereof.
20. The method of embodiment 1, wherein filtering comprises computationally filtering of the sequencing reads by bowtie2, Kraken, or any combination thereof programs.
21. The method of embodiment 1, wherein the protein database is the UniRef database.
22. The method of embodiment 1, wherein translating is accomplished by BLASTP, USEARCH, LAST, MMSeqs2, DIAMOND, or any combination thereof software packages.
23. The method of embodiment 2, wherein the mapping of the non-human proteins to the biochemical pathways is accomplished by mapping non-human proteins to KEGG, MetaCyc, PANTHER Pathway, PathBank or any combination thereof databases.
24. The method of embodiment 2, wherein the biochemical pathways are generated with the software package MinPath.
25. A method of providing a determination of the presence or lack thereof cancer of a subject, the method comprising:
   (a) sequencing a nucleic acid compositions of a subject's biological sample thereby generating sequencing reads;
   (b) filtering the sequencing reads with a genome database to produce a set of filtered non-human sequencing reads;
   (c) translating the non-human sequencing reads to non-human proteins;
   (d) mapping the non-human proteins to a protein database, thereby producing a set of protein database associations; and
   (e) providing a determination of the presence or lack thereof cancer of the subject as an output of a trained model when the trained model is provided an input of the set protein database associations.
26. The method of embodiment 25, wherein the set of protein database associations comprises a set of functional genes, biochemical pathways, or any combination thereof.
27. The method of embodiment 25, further comprising decontaminating the filtered non-human sequencing reads prior to (c) to remove contaminant non-human sequencing reads.
28. The method of embodiment 25, wherein translating is completed in silico.
29. The method of embodiment 25, wherein the biological sample is a tissue, liquid biopsy sample, or any combination thereof.
30. The method of embodiment 25, wherein the subject is human or a non-human mammal.
31. The method of embodiment 25, wherein the biological sample comprises a nucleic acid composition, wherein the nucleic acid composition comprises DNA, RNA, cell-free DNA, cell-free RNA, exosomal DNA, exosomal RNA, or any combination thereof.
32. The method of embodiment 25, wherein the genome database is a human genome database.
33. The method of embodiment 25, wherein the trained model is trained with a set of functional gene and biochemical pathway abundances that are present or absent with a characteristic abundance for a cancer of interest.
34. The method of embodiment 25, wherein the non-human sequences originate from bacterial, archaeal, fungal, viral, or any combination thereof origins of life.
35. The method of embodiment 25, wherein the trained model is configured to determine a category or tissue-specific location of the cancer of the subject.
36. The method of embodiment 25, wherein the trained model is configured to determine one or more types of the cancer of the subject.
37. The method of embodiment 36, wherein the trained model is configured to determine one or more subtypes of the cancer of the subject.
38. The method of embodiment 25, wherein the trained model is configured to determine a stage of a cancer of the subject, cancer prognosis of the subject, or any combination thereof.
39. The method of embodiment 25, wherein the trained model is configured to determine the presence or lack thereof a cancer at a low-stage (stage I or stage II) tumor.
40. The method of embodiment 25, wherein the trained model is configured to determine an immunotherapy response of the subject when the subject is provided an immunotherapy.
41. The method of embodiment 25, further comprising outputting with the trained model a therapy for the subject to treat the subject's cancer, wherein the subject will respond with positive therapeutic efficacy when administered the therapy.
42. The method of embodiment 25, wherein the cancer of the subject comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.
43. The method of embodiment 29, wherein the liquid biopsy comprises: plasma, serum, whole blood, urine, cerebral spinal fluid, saliva, sweat, tears, exhaled breath condensate, or any combination thereof.
44. The method of embodiment 25, wherein filtering comprises computationally filtering of the sequencing reads by bowtie2, Kraken, or any combination thereof programs.
45. The method of embodiment 25, wherein the protein database is the UniRef database.
46. The method of embodiment 25, wherein translating is accomplished by BLASTP, USEARCH, LAST, MMSeqs2, DIAMOND, or any combination thereof software packages.
47. The method of embodiment 26, wherein the mapping of the non-human proteins to the biochemical pathways is accomplished by mapping non-human proteins to KEGG, MetaCyc, PANTHER Pathway, PathBank or any combination thereof databases.
48. The method of embodiment 26, wherein the biochemical pathways are generated with the software package MinPath.
49. A method of training a model configured to determine the presence or lack thereof cancer of a subject, the method comprising:
   (a) providing a dataset comprising nucleic acid sequencing reads of a first set of one or more subjects' nucleic acid compositions and a corresponding one or more cancers of the first set of one or more subjects;
   (b) filtering the nucleic acid sequencing reads with a build of a genome database to generate non-human sequencing reads;
   (c) translating the non-human sequencing reads to non-human proteins;
   (d) mapping the non-human proteins to a protein database, thereby producing a set of protein database associations; and
   (e) training a model with the set of protein database associations and the corresponding one or more cancer states of the first set of one or more subjects, thereby generating a trained model configured to determine the presence or lack thereof cancer of a second set of one or more subjects.
50. The method of embodiment 49, wherein the set of protein database associations comprises a set of functional genes, biochemical pathways, or any combination thereof.
51. The method of embodiment 49, further comprising decontaminating the filtered non-human sequencing reads prior to (c) to remove contaminant non-human sequencing reads.
52. The method of embodiment 49, wherein translating is completed in silico.
53. The method of embodiment 49, wherein the biological sample is a tissue, liquid biopsy sample or any combination thereof.
54. The method of embodiment 49, wherein the first set, second set, or any combination thereof one or more subjects are human or a non-human mammal.
55. The method of embodiment 49, wherein the biological sample comprises a nucleic acid composition, wherein the nucleic acid composition comprises DNA, RNA, cell-free DNA, cell-free RNA, exosomal DNA, exosomal RNA, or any combination thereof.
56. The method of embodiment 49, wherein the genome database is a human genome database.
57. The method of embodiment 49, wherein the trained model is trained with a set of functional gene and biochemical pathway abundances that are present or absent with a characteristic abundance for a cancer of interest.
58. The method of embodiment 49, wherein the non-human sequences originate from bacterial, archaeal, fungal, viral, or any combination thereof origins of life.
59. The method of embodiment 49, wherein the trained model is configured to determine a category or tissue-specific location of the second set of one or more subjects' cancer.
60. The method of embodiment 49, wherein the trained model is configured to determine one or more types of the second set of one or more subjects' cancer.
61. The method of embodiment 60, wherein the trained model is configured to determine one or more subtypes of the second set of one or more subjects' cancer. 62. The method of embodiment 49, wherein the trained model is configured to determine a stage of the second set of one or more subjects' cancer, cancer prognosis, or any combination thereof.
63. The method of embodiment 49, wherein the trained is configured to determine the presence or lack thereof the second set of one or more subjects' cancer at a low-stage (stage I or stage II) tumor.
64. The method of embodiment 49, wherein the trained model is configured to determine an immunotherapy response of the subject when the subject is provided an immunotherapy.
65. The method of embodiment 49, further comprising outputting with the trained model a therapy to treat the second set of one or more subjects' cancer, wherein the second set of one or more subjects will respond with positive therapeutic efficacy when administered the therapy.
66. The method of embodiment 49, wherein the first and second set of one or more subjects' cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.
67. The method of embodiment 53, wherein the liquid biopsy comprises: plasma, serum, whole blood, urine, cerebral spinal fluid, saliva, sweat, tears, exhaled breath condensate, or any combination thereof.
68. The method of embodiment 49, wherein filtering comprises computationally filtering of the sequencing reads by bowtie2, Kraken, or any combination thereof programs.
69. The method of embodiment 49, wherein the protein database is the UniRef database.
70. The method of embodiment 49, wherein translating is accomplished by BLASTP, USEARCH, LAST, MMSeqs2, DIAMOND, or any combination thereof software packages.
71. The method of embodiment 50, wherein the mapping of the non-human proteins to the biochemical pathways is accomplished by mapping non-human proteins to KEGG, MetaCyc, PANTHER Pathway, PathBank or any combination thereof databases.
72. The method of embodiment 50, wherein the biochemical pathways are generated with the software package MinPath.
73. The method of embodiment 51, wherein the dataset further comprises a corresponding previous or current treatment administered to the first set of one or more subjects.
74. The method of embodiment 73, wherein the dataset further comprises a treatment efficacy of the first set of one or more subjects' previous or current treatment administration.
75. A computer-implemented method for utilizing a trained predictive model to provide a therapeutic treatment prediction for one or more subjects, the method comprising:
   (a) receiving a first set of one or more subjects' nucleic acid sequencing reads of a biological sample and corresponding cancer classification;
   (b) filtering the nucleic acid sequencing reads with a build of a genome database to generate non-human sequencing reads;
   (c) translating the non-human sequencing reads to non-human proteins;
   (d) mapping the non-human proteins to a protein database, thereby producing a set of protein database associations; and
   (e) utilizing a trained predictive model to provide a treatment prediction for the first set of one or more subjects when the set of protein database associations are provided as an input to the trained predictive model.
76. The method of embodiment 75, wherein the trained predictive model is trained on a second set of one or more subjects' nucleic acid sequencing reads of a biological sample, corresponding cancer classification, corresponding treatment administered, corresponding treatment response, or any combination thereof.
77. The method of embodiment 76, wherein the second set of one or more subjects are different than the first set of one or more subjects.
78. The method of embodiment 75, wherein the set of protein database associations comprises a set of functional genes, biochemical pathways, or any combination thereof.
79. The method of embodiment 75, further comprising decontaminating the filtered non-human sequencing reads prior to (c) to remove contaminant non-human sequencing reads.
80. The method of embodiment 75, wherein translating is completed in silico.
81. The method of embodiment 75, wherein the biological sample is a tissue, liquid biopsy sample or any combination thereof.
82. The method of embodiment 75, wherein the first set of one or more subjects are human or a non-human mammal.
83. The method of embodiment 75, wherein the biological sample nucleic acid composition comprises DNA, RNA, cell-free DNA, cell-free RNA, exosomal DNA, exosomal RNA, or any combination thereof.
84. The method of embodiment 75, wherein the genome database is a human genome database.
85. The method of embodiment 75, wherein the non-human sequences originate from bacterial, archaeal, fungal, viral, or any combination thereof origins of life.
86. The method of embodiment 75, wherein the treatment prediction comprises an immunotherapy response of the first set of one or more subjects when the first set of one or more subjects are administered an immunotherapy.
87. The method of embodiment 75, wherein the treatment prediction comprises a therapeutic efficacy that the first set of one or more subjects will respond with positive efficacy.
88. The method of embodiment 75, wherein the cancer classification comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.
89. The method of embodiment 79, wherein the liquid biopsy comprises: plasma, serum, whole blood, urine, cerebral spinal fluid, saliva, sweat, tears, exhaled breath condensate, or any combination thereof.
90. The method of embodiment 75, wherein filtering comprises computationally filtering of the sequencing reads by bowtie2, Kraken, or any combination thereof programs.
91. The method of embodiment 75, wherein the protein database is the UniRef database.
92. The method of embodiment 75, wherein translating is accomplished by BLASTP, USEARCH, LAST, MMSeqs2, DIAMOND, or any combination thereof software packages.
93. The method of embodiment 76, wherein the mapping of the non-human proteins to the biochemical pathways is accomplished by mapping non-human proteins to KEGG, MetaCyc, PANTHER Pathway, PathBank or any combination thereof databases.
94. The method of embodiment 76, wherein the biochemical pathways are generated with the software package MinPath.
95. A method of changing a subject's cancer treatment with a trained predictive model, the method comprising:
   (a) providing one or more sequencing reads of a subject's biological sample with cancer, cancer type, and treatment administered to treat the cancer;
   (b) filtering the sequencing reads with a genome database to produce a set of filtered non-human sequencing reads;
   (c) translating the non-human sequencing reads to non-human proteins;
   (d) mapping the non-human proteins to a protein database, thereby producing a set of protein database associations; and
   (e) changing the subject's cancer treatment when the treatment administered differs from a treatment recommendation outputted by a trained predictive model when inputted with the set of protein database associations.
96. The method of embodiment 95, wherein the trained predictive model is trained on a second set of one or more subjects' nucleic acid sequencing reads of a biological sample, corresponding cancer classification, corresponding treatment administered, corresponding treatment response, or any combination thereof.
97. The method of embodiment 96, wherein the second set of one or more subjects are different than the first set of one or more subjects.
98. The method of embodiment 95, wherein the set of protein database associations comprises a set of functional genes, biochemical pathways, or any combination thereof.
99. The method of embodiment 95, further comprising decontaminating the filtered non-human sequencing reads prior to (c) to remove contaminant non-human sequencing reads.
100. The method of embodiment 95, wherein translating is completed in silico.
101. The method of embodiment 95, wherein the biological sample is a tissue, liquid biopsy sample or any combination thereof.
102. The method of embodiment 95, wherein the subject is human or a non-human mammal.
103. The method of embodiment 95, wherein the biological sample nucleic acid composition comprises DNA, RNA, cell-free DNA, cell-free RNA, exosomal DNA, exosomal RNA, or any combination thereof.
104. The method of embodiment 95, wherein the genome database is a human genome database.
105. The method of embodiment 95, wherein the non-human sequences originate from bacterial, archaeal, fungal, viral, or any combination thereof origins of life.
106. The method of embodiment 95, wherein the treatment recommendation comprises an immunotherapy response of the subject when the subject is administered an immunotherapy.
107. The method of embodiment 95, wherein the treatment recommendation comprises a therapeutic that the subject will respond with positive efficacy.
108. The method of embodiment 95, wherein the subject's cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.
109. The method of embodiment 101, wherein the liquid biopsy comprises: plasma, serum, whole blood, urine, cerebral spinal fluid, saliva, sweat, tears, exhaled breath condensate, or any combination thereof.
110. The method of embodiment 95, wherein filtering comprises computationally filtering of the sequencing reads by bowtie2, Kraken, or any combination thereof programs.
111. The method of embodiment 95, wherein the protein database is the UniRef database.
112. The method of embodiment 95, wherein translating is accomplished by BLASTP, USEARCH, LAST, MMSeqs2, DIAMOND, or any combination thereof software packages.
113. The method of embodiment 96, wherein the mapping of the non-human proteins to the biochemical pathways is accomplished by mapping non-human proteins to KEGG, MetaCyc, PANTHER Pathway, PathBank or any combination thereof databases.
114. The method of embodiment 96, wherein the biochemical pathways are generated with the software package MinPath.

What is claimed:

1. A method of determining the presence or lack thereof cancer of a subject, the method comprising:
(a) providing one or more sequencing reads of a subject's biological sample;
(b) filtering the sequencing reads with a genome database to produce a set of filtered non-human sequencing reads;
(c) translating the non-human sequencing reads to non-human proteins;
(d) mapping the non-human proteins to a protein database, thereby producing a set of protein database associations; and
(e) determining the presence or lack thereof cancer of the subject as an output to the trained model when the trained model is provided an input of the set of protein database associations.

2. The method of claim 1, wherein the set of protein database associations comprises a set of functional genes, biochemical pathways, or any combination thereof.

3. The method of claim 1, further comprising decontaminating the filtered non-human sequencing reads prior to (c) to remove contaminant non-human sequencing reads.

4. The method of claim 1, wherein translating is completed in silico.

5. The method of claim 1, wherein the biological sample is a tissue, liquid biopsy, or any combination thereof.

6. The method of claim 1, wherein the subject is human or a non-human mammal.

7. The method of claim 1, wherein the biological sample comprises a nucleic acid composition, wherein the nucleic acid composition comprises DNA, RNA, cell-free DNA, cell-free RNA, exosomal DNA, exosomal RNA, or any combination thereof.

8. The method of claim 1, wherein the genome database is a human genome database.

9. The method of claim 1, wherein the trained model is trained with a set of functional gene and biochemical pathway abundances that are present or absent with a characteristic abundance for a cancer of interest.

10. The method of claim 1, wherein the non-human sequences originate from bacterial, archaeal, fungal, viral, or any combination thereof origins of life.

11. The method of claim 1, wherein the trained model is configured to determine a category or tissue-specific location of the cancer of the subject.

12. The method of claim 1, wherein the trained model is configured to determine one or more types of cancer of the subject.

13. The method of claim 12, wherein the trained model is configured to determine one or more subtypes of the cancer of the subject.

14. The method of claim 1, wherein the trained model is configured to determine a stage of cancer of the subject, cancer prognosis of the subject, or any combination thereof.

15. The method of claim 1, wherein the trained model is configured to determine the presence or lack thereof cancer at a low-stage (stage I or stage II) tumor.

16. The method of claim 1, wherein the trained model is configured to determine an immunotherapy response of the subject when the subject is provided the immunotherapy.

17. The method of claim 1, further comprising outputting with the trained model a therapy for the subject to treat the subject's cancer, wherein the subject will respond with positive therapeutic efficacy when administered the therapeutic.

18. The method of claim 1, wherein the cancer of the subject comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

19. The method of claim 5, wherein the liquid biopsy comprises: plasma, serum, whole blood, urine, cerebral spinal fluid, saliva, sweat, tears, exhaled breath condensate, or any combination thereof.

20. The method of claim 1, wherein filtering comprises computationally filtering of the sequencing reads by bowtie2, Kraken, or any combination thereof programs.

21. The method of claim 1, wherein the protein database is the UniRef database.

22. The method of claim 1, wherein translating is accomplished by BLASTP, USEARCH, LAST, MMSeqs2, DIAMOND, or any combination thereof software packages.

23. The method of claim 2, wherein the mapping of the non-human proteins to the biochemical pathways is accomplished by mapping non-human proteins to KEGG, MetaCyc, PANTHER Pathway, PathBank or any combination thereof databases.

24. The method of claim 2, wherein the biochemical pathways are generated with the software package MinPath.

25. A method of providing a determination of the presence or lack thereof cancer of a subject, the method comprising:
(a) sequencing a nucleic acid compositions of a subject's biological sample thereby generating sequencing reads;
(b) filtering the sequencing reads with a genome database to produce a set of filtered non-human sequencing reads;
(c) translating the non-human sequencing reads to non-human proteins;
(d) mapping the non-human proteins to a protein database, thereby producing a set of protein database associations; and
(e) providing a determination of the presence or lack thereof cancer of the subject as an output of a trained model when the trained model is provided an input of the set protein database associations.

26. The method of claim 25, wherein the set of protein database associations comprises a set of functional genes, biochemical pathways, or any combination thereof.

27. The method of claim 25, further comprising decontaminating the filtered non-human sequencing reads prior to (c) to remove contaminant non-human sequencing reads.

28. The method of claim 25, wherein translating is completed in silico.

29. The method of claim 25, wherein the biological sample is a tissue, liquid biopsy sample, or any combination thereof.

30. The method of claim 25, wherein the subject is human or a non-human mammal.

31. The method of claim 25, wherein the biological sample comprises a nucleic acid composition, wherein the nucleic acid composition comprises DNA, RNA, cell-free DNA, cell-free RNA, exosomal DNA, exosomal RNA, or any combination thereof.

32. The method of claim 25, wherein the genome database is a human genome database.

33. The method of claim 25, wherein the trained model is trained with a set of functional gene and biochemical pathway abundances that are present or absent with a characteristic abundance for a cancer of interest.

34. The method of claim 25, wherein the non-human sequences originate from bacterial, archaeal, fungal, viral, or any combination thereof origins of life.

35. The method of claim 25, wherein the trained model is configured to determine a category or tissue-specific location of the cancer of the subject.

36. The method of claim 25, wherein the trained model is configured to determine one or more types of the cancer of the subject.

37. The method of claim 36, wherein the trained model is configured to determine one or more subtypes of the cancer of the subject.

38. The method of claim 25, wherein the trained model is configured to determine a stage of a cancer of the subject, cancer prognosis of the subject, or any combination thereof.

39. The method of claim 25, wherein the trained model is configured to determine the presence or lack thereof a cancer at a low-stage (stage I or stage II) tumor.

40. The method of claim 25, wherein the trained model is configured to determine an immunotherapy response of the subject when the subject is provided an immunotherapy.

41. The method of claim 25, further comprising outputting with the trained model a therapy for the subject to treat the subject's cancer, wherein the subject will respond with positive therapeutic efficacy when administered the therapy.

42. The method of claim 25, wherein the cancer of the subject comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

43. The method of claim 29, wherein the liquid biopsy comprises: plasma, serum, whole blood, urine, cerebral spinal fluid, saliva, sweat, tears, exhaled breath condensate, or any combination thereof.

44. The method of claim 25, wherein filtering comprises computationally filtering of the sequencing reads by bowtie2, Kraken, or any combination thereof programs.

45. The method of claim 25, wherein the protein database is the UniRef database.

46. The method of claim 25, wherein translating is accomplished by BLASTP, USEARCH, LAST, MMSeqs2, DIAMOND, or any combination thereof software packages.

47. The method of claim 26, wherein the mapping of the non-human proteins to the biochemical pathways is accomplished by mapping non-human proteins to KEGG, MetaCyc, PANTHER Pathway, PathBank or any combination thereof databases.

48. The method of claim 26, wherein the biochemical pathways are generated with the software package MinPath.

49. A method of training a model configured to determine the presence or lack thereof cancer of a subject, the method comprising:
(a) providing a dataset comprising nucleic acid sequencing reads of a first set of one or more subjects' nucleic acid compositions and a corresponding one or more cancers of the first set of one or more subjects;
(b) filtering the nucleic acid sequencing reads with a build of a genome database to generate non-human sequencing reads;
(c) translating the non-human sequencing reads to non-human proteins;
(d) mapping the non-human proteins to a protein database, thereby producing a set of protein database associations; and
(e) training a model with the set of protein database associations and the corresponding one or more cancer states of the first set of one or more subjects, thereby generating a trained model configured to determine the presence or lack thereof cancer of a second set of one or more subjects.

50. The method of claim 49, wherein the set of protein database associations comprises a set of functional genes, biochemical pathways, or any combination thereof.

51. The method of claim 49, further comprising decontaminating the filtered non-human sequencing reads prior to (c) to remove contaminant non-human sequencing reads.

52. The method of claim 49, wherein translating is completed in silico.

53. The method of claim 49, wherein the biological sample is a tissue, liquid biopsy sample or any combination thereof.

54. The method of claim 49, wherein the first set, second set, or any combination thereof one or more subjects are human or a non-human mammal.

55. The method of claim 49, wherein the biological sample comprises a nucleic acid composition, wherein the nucleic acid composition comprises DNA, RNA, cell-free DNA, cell-free RNA, exosomal DNA, exosomal RNA, or any combination thereof.

56. The method of claim 49, wherein the genome database is a human genome database.

57. The method of claim 49, wherein the trained model is trained with a set of functional gene and biochemical pathway abundances that are present or absent with a characteristic abundance for a cancer of interest.

58. The method of claim 49, wherein the non-human sequences originate from bacterial, archaeal, fungal, viral, or any combination thereof origins of life.

59. The method of claim 49, wherein the trained model is configured to determine a category or tissue-specific location of the second set of one or more subjects' cancer.

60. The method of claim 49, wherein the trained model is configured to determine one or more types of the second set of one or more subjects' cancer.

61. The method of claim 60, wherein the trained model is configured to determine one or more subtypes of the second set of one or more subjects' cancer.

62. The method of claim 49, wherein the trained model is configured to determine a stage of the second set of one or more subjects' cancer, cancer prognosis, or any combination thereof.

63. The method of claim 49, wherein the trained is configured to determine the presence or lack thereof the second set of one or more subjects' cancer at a low-stage (stage I or stage II) tumor.

64. The method of claim 49, wherein the trained model is configured to determine an immunotherapy response of the subject when the subject is provided an immunotherapy.

65. The method of claim 49, further comprising outputting with the trained model a therapy to treat the second set of one or more subjects' cancer, wherein the second set of one or more subjects will respond with positive therapeutic efficacy when administered the therapy.

66. The method of claim 49, wherein the first and second set of one or more subjects' cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

67. The method of claim 53, wherein the liquid biopsy comprises: plasma, serum, whole blood, urine, cerebral spinal fluid, saliva, sweat, tears, exhaled breath condensate, or any combination thereof.

68. The method of claim 49, wherein filtering comprises computationally filtering of the sequencing reads by bowtie2, Kraken, or any combination thereof programs.

69. The method of claim 49, wherein the protein database is the UniRef database.

70. The method of claim 49, wherein translating is accomplished by BLASTP, USEARCH, LAST, MMSeqs2, DIAMOND, or any combination thereof software packages.

71. The method of claim 50, wherein the mapping of the non-human proteins to the biochemical pathways is accomplished by mapping non-human proteins to KEGG, MetaCyc, PANTHER Pathway, PathBank or any combination thereof databases.

72. The method of claim 50, wherein the biochemical pathways are generated with the software package MinPath.

73. The method of claim 51, wherein the dataset further comprises a corresponding previous or current treatment administered to the first set of one or more subjects.

74. The method of claim 73, wherein the dataset further comprises a treatment efficacy of the first set of one or more subjects' previous or current treatment administration.

75. A computer-implemented method for utilizing a trained predictive model to provide a therapeutic treatment prediction for one or more subjects, the method comprising:
(f) receiving a first set of one or more subjects' nucleic acid sequencing reads of a biological sample and corresponding cancer classification;
(g) filtering the nucleic acid sequencing reads with a build of a genome database to generate non-human sequencing reads;
(h) translating the non-human sequencing reads to non-human proteins;
(i) mapping the non-human proteins to a protein database, thereby producing a set of protein database associations; and
(j) utilizing a trained predictive model to provide a treatment prediction for the first set of one or more subjects when the set of protein database associations are provided as an input to the trained predictive model.

76. The method of claim 75, wherein the trained predictive model is trained on a second set of one or more subjects' nucleic acid sequencing reads of a biological sample, corresponding cancer classification, corresponding treatment administered, corresponding treatment response, or any combination thereof.

77. The method of claim 76, wherein the second set of one or more subjects are different than the first set of one or more subjects.

78. The method of claim 75, wherein the set of protein database associations comprises a set of functional genes, biochemical pathways, or any combination thereof.

79. The method of claim 75, further comprising decontaminating the filtered non-human sequencing reads prior to (c) to remove contaminant non-human sequencing reads.

80. The method of claim 75, wherein translating is completed in silico.

81. The method of claim 75, wherein the biological sample is a tissue, liquid biopsy sample or any combination thereof.

82. The method of claim 75, wherein the first set of one or more subjects are human or a non-human mammal.

83. The method of claim 75, wherein the biological sample nucleic acid composition comprises DNA, RNA, cell-free DNA, cell-free RNA, exosomal DNA, exosomal RNA, or any combination thereof.

84. The method of claim 75, wherein the genome database is a human genome database.

85. The method of claim 75, wherein the non-human sequences originate from bacterial, archaeal, fungal, viral, or any combination thereof origins of life.

86. The method of claim 75, wherein the treatment prediction comprises an immunotherapy response of the first set of one or more subjects when the first set of one or more subjects are administered an immunotherapy.

87. The method of claim 75, wherein the treatment prediction comprises a therapeutic efficacy that the first set of one or more subjects will respond with positive efficacy.

88. The method of claim 75, wherein the cancer classification comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

89. The method of claim 79, wherein the liquid biopsy comprises: plasma, serum, whole blood, urine, cerebral spinal fluid, saliva, sweat, tears, exhaled breath condensate, or any combination thereof.

90. The method of claim 75, wherein filtering comprises computationally filtering of the sequencing reads by bowtie2, Kraken, or any combination thereof programs.

91. The method of claim 75, wherein the protein database is the UniRef database.

92. The method of claim 75, wherein translating is accomplished by BLASTP, USEARCH, LAST, MMSeqs2, DIAMOND, or any combination thereof software packages.

93. The method of claim 76, wherein the mapping of the non-human proteins to the biochemical pathways is accomplished by mapping non-human proteins to KEGG, MetaCyc, PANTHER Pathway, PathBank or any combination thereof databases.

94. The method of claim 76, wherein the biochemical pathways are generated with the software package MinPath.

95. A method of changing a subject's cancer treatment with a trained predictive model, the method comprising:
(a) providing one or more sequencing reads of a subject's biological sample with cancer, cancer type, and treatment administered to treat the cancer;
(b) filtering the sequencing reads with a genome database to produce a set of filtered non-human sequencing reads;
(c) translating the non-human sequencing reads to non-human proteins;
(d) mapping the non-human proteins to a protein database, thereby producing a set of protein database associations; and
(e) changing the subject's cancer treatment when the treatment administered differs from a treatment recommendation outputted by a trained predictive model when inputted with the set of protein database associations.

96. The method of claim 95, wherein the trained predictive model is trained on a second set of one or more subjects' nucleic acid sequencing reads of a biological sample, corresponding cancer classification, corresponding treatment administered, corresponding treatment response, or any combination thereof.

97. The method of claim 96, wherein the second set of one or more subjects are different than the first set of one or more subjects.

98. The method of claim 95, wherein the set of protein database associations comprises a set of functional genes, biochemical pathways, or any combination thereof.

99. The method of claim 95, further comprising decontaminating the filtered non-human sequencing reads prior to (c) to remove contaminant non-human sequencing reads.

100. The method of claim 95, wherein translating is completed in silico.

101. The method of claim 95, wherein the biological sample is a tissue, liquid biopsy sample or any combination thereof.

102. The method of claim 95, wherein the subject is human or a non-human mammal.

103. The method of claim 95, wherein the biological sample nucleic acid composition comprises DNA, RNA, cell-free DNA, cell-free RNA, exosomal DNA, exosomal RNA, or any combination thereof.

104. The method of claim 95, wherein the genome database is a human genome database.

105. The method of claim 95, wherein the non-human sequences originate from bacterial, archaeal, fungal, viral, or any combination thereof origins of life.

106. The method of claim 95, wherein the treatment recommendation comprises an immunotherapy response of the subject when the subject is administered an immunotherapy.

107. The method of claim 95, wherein the treatment recommendation comprises a therapeutic that the subject will respond with positive efficacy.

108. The method of claim 95, wherein the subject's cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

109. The method of claim 101, wherein the liquid biopsy comprises: plasma, serum, whole blood, urine, cerebral spinal fluid, saliva, sweat, tears, exhaled breath condensate, or any combination thereof.

110. The method of claim 95, wherein filtering comprises computationally filtering of the sequencing reads by bowtie2, Kraken, or any combination thereof programs.

111. The method of claim 95, wherein the protein database is the UniRef database.

112. The method of claim 95, wherein translating is accomplished by BLASTP, USEARCH, LAST, MMSeqs2, DIAMOND, or any combination thereof software packages.

113. The method of claim 96, wherein the mapping of the non-human proteins to the biochemical pathways is accomplished by mapping non-human proteins to KEGG, MetaCyc, PANTHER Pathway, PathBank or any combination thereof databases.

114. The method of claim 96, wherein the biochemical pathways are generated with the software package MinPath.
